SEQUENCE LISTING (18 sequences)

SEQ ID NO: 1 - DNA, 589 bp, Artificial
  Vector Element
  Misc._Feature (1)..(589): Name/Key:: Human Cytolomegavirus Early Promoter/Enhancer (CMV).
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 2 - DNA, 588 bp, Artificial
  Vector Element
  Misc._Feature (1)..(588): Name/Key: Internal Ribosome Entry Site (IRES)
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 3 - DNA, 598 bp, Artificial
  Vector Element
  Misc._Feature (1)..(598): Name/Key: Woodchuck Hepatitus Virus Posttranscriptional Regulatory Element (WPRE)
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 4 - DNA, 236 bp, Artificial
  Vector Element
  Misc._Feature (1)..(236): Name/Key: Simian Virus 40 Polyadenylation Signal (SV40 Poly A)
agctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgg

SEQ ID NO: 5 - DNA, 208 bp, Artificial
  Vector Element
  Misc._Feature (1)..(208): Name/Key: Bovine Growth Hormone Polyadenylation Signal (BGH Poly A)
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 6 - skipped

SEQ ID NO: 7 - protein, 136 aa, Human
  Misc._Feature (1)..(136): Name/Key: J Chain Without Signaling Peptide
EDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 8 - protein, 189 aa, Human
  Misc._Feature (11)..(189): Name/Key: Marginal Zone B-1 Cell Specific Protein (MZB1)
MRLSLPLLLLLLGAWAIPGGLGDRAPLTATAPQLDDEEMYSAHMPAHLRCDACRAVAYQMWQNLAKAETKLHTSNSGGRRELSELVYTDVLDRSCSRNWQDYGVREVDQVKRLTGPGLSEGPEPSISVMVTGGPWPTRLSRTCLHYLGEFGEDQIYEAHQQGRGALEALLCGGPQGACSEKVSATREEL

SEQ ID NO: 9 - DNA, 117 bp, Artificial
  Vector Element
  Misc._Feature (1)..(117): Name/Key: Intron I117 5′ Untranslated Region (5′ UTR)
gtaagtgatatctactagatttatcaaaaagagtgttgacttgtgagcgctcacaattgatacttagattcatcgagagggacacgtcgactactaaccttcttctctttcctacag

SEQ ID NO: 10 - protein, 4 aa, Artificial
  Vector Element
  Misc._Feature (1)..(4): Name/Key: Consensus Sequence of Furin Cleavage Site (Furin Cleavage Site)
  Misc._Feature (2)..(2): Xaa = any amino acid
RXKR

SEQ ID NO: 11 - protein, 159 aa, Human
  Misc._Feature (1)..(159): Name/Key: J Chain with Signaling Peptide
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 12 - protein, 5 aa, Artificial
  Vector Element
  Misc._Feature (1)..(5): Name/Key: Consensus Sequence of Furin Cleavage Site 2 (Furin Cleavage Site)
  Misc._Feature (2)..(2): Xaa = any amino acid
RXKRR

SEQ ID NO: 13 - protein, 6 aa, Artificial
  Vector Element
  Misc._Feature (1)..(6): Name/Key: Consensus Sequence of Furin Cleavage Site 3 (Furin Cleavage Site)
  Misc._Feature (2)..(2): Xaa = any amino acid
RXRYKR

SEQ ID NO: 14 - protein, 6 aa, Artificial
  Vector Element
  Misc._Feature (1)..(6): Name/Key: Consensus Sequence of Furin Cleavage Site 4 (Furin Cleavage Site)
  Misc._Feature (2)..(2): Xaa = any amino acid
RXRFKR

SEQ ID NO: 15 - protein, 25 aa, Artificial
  Vector Element
  Misc._Feature (1)..(25): Name/Key: Foot and Mouth Disease Virus 2A Self Cleaving/Self Processing Peptide (F2A) or (2A)
GSGVKQTLNFDLLKLAGDVESNPGP

SEQ ID NO: 16 - protein, 19 aa, Artificial
  Vector Element
  Misc._Feature (1)..(19): Name/Key: Porcine Teschovirus 2A Self Cleaving/Self Processing Peptide (P2A) or (2A)
ATNFSLLKQAGDVEENPGP

SEQ ID NO: 17 - protein, 18 aa, Artificial
  Vector Element
  Misc._Feature (1)..(18): Name/Key: Thosea Asigna Virus-2A Self Cleaving/Self Processing Peptide (T2A) or (2A)
EGRGSLLTCGDVEENPGP

SEQ ID NO: 18 - DNA, 6 bp, Human
  Misc._Feature (1)..(6): From Page 42 of the Specification
  Misc._Feature (1)..(6): Name/Key: Kozak Sequence
gccacc

(19) United States
(12) Patent Application Publication
(10) Pub. No.: US 2022/0064265 A1
(43) Pub. Date: Mar. 3, 2022

(54) Episomal Expression of Potent Immunoglobulins Derived from Human Blood or Convalescent Plasma to Enable Short term Vaccination / Immunization to COVID, COVID-19 and Mutants and Other Pandemic and non-Pandemic Viruses designed from Rapid FDA approval.

(71) Applicant: Roger B Swartz, Clifton Park, NY (US)

(72) Inventor: Roger B Swartz, Clifton Park, NY (US)

(21) Appl. No.: 16/995,829

(22) Filed: Aug. 18, 2020

(51) Int. Cl. C07K 16/10; C12N 15/85; G01N 33/68

(52) U.S. Cl. CPC C07K 16/10; A61K 48/00; G01N 33/6854; C12N 15/85

(57) ABSTRACT

The present invention provides methods, immunoglobulin compositions and vector constructs as a general approach to provide episomal based immune protection from the 2019 novel coronavirus (COVID-19), its variants/mutants and other pandemic and even non-pandemic viruses. The immunoglobulin compositions include the heavy chain variable, diversity and joining (VDJ or Variable Heavy Region genes) segment immunoglobulin DNA and/or polypeptide sequence from humans identified to have developed high affinity immunoglobulins (ideally antibodies with nanomolar to picomolar dissociation constants to virus proteins with additional emphasis on cell surface proteins and further emphasis on the Spike protein as related to COVID-19) against the virus of interest and either to use the exact immunoglobulin composition identified from the donor or to combine that variable immunoglobulin region for both heavy and light chains with a non-divergent well-conserved amino acid sequence for the constant regions especially, Hinge region, Constant Heavy 2 (C_{H}2) and Constant Heavy 3 (C_{H}3) for the immunoglobulin heavy chain polypeptide with optional use of donor based Constant Heavy 1 (C_{H}1) or non-divergent well conserved C_{H}1 heavy chain constant region and optional use of hinge region peptides. The immunoglobulin light chain will use either entirely donor based amino acid sequence or donor based light chain variable and joining (VJ or Variable light region genes) segments immunoglobulin polypeptide sequence with a well-conserved non-divergent constant light (C_{L}) chain region for immunoglobulin Kappa locus (κ) or immunoglobulin lambda locus (λ) light chain. The resulting antibodies can either be used as a monoclonal or polyclonal mix of (Immunoglobulin Class G subclass1) IgG1, IgG3 and other subclasses, IgA1 monomer and IgA2 monomer and dimeric IgA1 (dIgA1) immunoglobulins (as identified by the potency of associated memory B-cells) to be expressed via intramuscular administration, intravenous or proximal to lymph nodes. The immunoglobulins will be expressed in the vaccine/immunization recipient via an episome. The vector will be ideally delivered in a recombinant Adeno Associated Virus (rAAV) with preference for AAV serotype 8 (AAV8) containing a single-stranded Deoxyribonucleic acid (ssDNA) non-viral vector or lentivirus virion containing double stranded DNA as a non-viral vector. A single non-viral vector will code for the entire immunoglobulin and J-chain expression for dIgA1 where expression will occur with a single start codon and stop codon for the amino acid sequence and in some embodiments a second start codon for J chain expression. The specific DNA of the immune donor can be identified as follows: Cluster of Differentiation 27+ (CD27+) IgG+ and CD27+ IgA+ memory B cells or other CD memory B-cells will be isolated from serum using established methods. Each resulting isotype of memory B-cell will be subjected to a competitive binding assay using flow cytometry methods such as Fluorescence Activated Cell Sorting (FACS) to identify the memory B-cells with the greatest binding affinity to the COVID-19 antigens of interest. Isolated memory B-cells will have their DNA sequenced to identify the genetic sequence of their cell surface IgG+ or IgA+ receptor. That information and potentially other sources of immunoglobulin genetic information will be used to create vector construct coding for antibodies to be further evaluative for potency and safety and then to be incorporated into a vector construct for episomal immunoglobulin expression. Episomes will be designed to express IgG1, IgG3, IgA1, IgA2 and dIgA1 with potent binding to COVID antigens or antigens of other viruses. A central part of this patent application is the method used to identify the high affinity immunoglobulins expressed by those that were exposed to COVID or other virus of interest.

### CROSS-REFERENCE TO RELATED APPLICATIONS

This present application claims priority to U.S. provisional patent application Ser. No. 63/008,844. The entire disclosure is included herein in its entirety at least by reference.

### REFERENCE TO AN ELECTRONIC SEQUENCE LISTING

In accordance with 37 CFR 1.52(e)(5) on May 28, 2021 the ASCII text filed named “Roger_Swartz_Sequence_listing-16_995_829_for_Upload.txt” was uploaded electronically to the EFS-web. The file was created on May 28, 2021 and the size of the file is 12,680 bytes (16 KB on Disk). The contents of file named “Roger_Swartz_Sequence_listing_16_995_829_for_Upload.txt” containing the sequence listing is herein incorporated by reference in its entirety.”

### REFERENCE TO SEQUENCE LISTING UPLOADED ELECTRONICALLY BY PDF

In accordance with 37 CFR 1.821(c) on May 28, 2021 the PDF filed named “Roger_B_Swartz_non-Provisional_Patent_Application_Sequence_Listings_16_995_829_Clean. pdf” was uploaded electronically to the EFS-Web. The file was created on May 26, 2021 and the size of the file is 121,944 bytes. The contents of file named “Roger_B_Swartz_non-Provisional_Patent_Application_Sequence_Listings_16_995_829_Clean.pdf” containing the sequence listing is herein incorporated by reference in its entirety.

### BACKGROUND OF THE INVENTION

### 1. Field of the Invention

The present invention is in the field of healthcare and biotechnology and pertains particularly to episomal expression of immunoglobulins, immunoglobulin fragments or immunoglobulin V-regions potent against the virus of interest that are naturally expressed in individuals that were infected from viruses such as COVID, COVID-19 and mutants and other pandemic viruses.

Specifically the immunoglobulin V-region and potentially constant region genetic sequence would be identified from CD27+ memory B-cell—expressing immunoglobulins with relatively high binding affinity for a virus antigen—of a human that have been naturally infected with the virus or mutant form of the virus of interest. Most likely those B-cells would be CD27+ IgG and IgA memory B-cells would bear cell surface immunoglobulins easily allowing for their isolation through cell sorting methods such as flow cytometry or fluorescence activated cell sorting (FACS) or magnetic pull down methods that incorporate biotinylation with an antigen of interest. The genetic information encoding for the monoclonal immunoglobulin expressed on the B-cells of interest will be identified. Vectors may be created that encode for those immunoglobulins to be evaluated. Additionally, the V-region DNA of the heavy (IgH) and light chain (IgL) of the immunoglobulins will be incorporated into a vector construct that could use either the constant region DNA identified in the B-cell expressing the identified immunoglobulin of interest or another source of genetic information may replace all or part of the constant region DNA that may also include isotype switching of constant region genetic information, mixes of two constant regions from two isotypes that may be accomplished with combinations of Fab and natural or engineered Fc domains or F(ab′)₂ and natural or engineered pFc′ domains. The episome may be delivered to include B-cells and muscle cells as well as other cells potentially. The episome may be delivered to the cells of interest via an adeno-associated virus, lentivirus based deliver system or a vesicle based delivery system.

### 2. Discussion of the State of the Art

Pandemic Viruses can cause serious health complications, present and future unknown health complications and catastrophic economic stress on a country with significant collateral damage such as increased unemployment, mental health challenges, school closures and bankruptcy. All these collateral damage examples have been experienced as a result of the 2019 novel Coronavirus (COVID-19) and mutants. There is also a constant threat of future pendemic and non-pandemic viruses that we do not fully understand and cannot estimate.

As a result of the ease of transmission and the threat of death and unknown health risks there has been an urgency to find a vaccine or immunization that will keep humans safe and allow them and society to resume activities as normal. The combined immune response and damage due to COVID-19 or its mutants once it breaches the epithelial lining has included the following: cytokine storm, Kawasaki disease, other forms of autoimmunity, organ damage, stroke and death. The long-term health consequences caused by COVID-19 infections are not known. Additionally, it is not clear the extent to which reinfection can bring about a more significant health risks. In order for an individual to effectively prevent COVID or other respiratory virus from infecting and crossing the epithelial barrier mucosal immunity is required. Mucosal immunity occurs in the upper respiratory tract, lungs and intestinal tract. Because of the complications and risks associated with COVID infecting and crossing the epithelial barrier the most ideal immunity to COVID-19 begins with an upper respiratory mucosal immunity as well as mucosal immunity in the lumen of the bronchi and increasingly finer bronchioles. Secretory immunoglobulin A (SIgA) is by far the most prevalent antibody in the human body and represents a major mode of defense in mucosal immunity. It is mucosal immunity that is required to greatly reduce the probability of cytokine storm for those at risk to COVID-19. It is mucosal immunity that is required to prevent COVID-19 and its mutants from crossing the epithelial barrier. It is mucosal immunity that can effectively prevent humans that get exposed to COVID-19 from passing it to others. In other words to stop the spread and harmful effects of COVID-19 and its mutants on all members of society including those at risk mucosal immunity is required. See e.g., Gianchecchi, E., et. al. 2019, Influenza Other Respir. Viruses. 13:429-437. Also, for a review on mucosal immunity see e.g., Pilette, C., et. al., 2001, European Respiratory Journal, 18:571-588. Also, see e.g., Terauchi, Y., et. al., 2018, Human vaccines & immunotherapeutics, 14:1351-1361.)

Combating viruses especially pandemic viruses that are highly contagious results in efforts to develop healthcare solutions to prevent infection from these viruses that may be classified as vaccines, immunizations, passive immunizations, polyclonal antibodies, monoclonal antibodies, modified antibodies. Because pandemic viruses do not come frequently the strategies employed for each new wave of pandemic viruses can include entirely new strategies that differ from an earlier pandemic. In the present COVID-19 and mutants pandemic has included two vaccination strategies include the use of weakened forms of the virus and the use of messenger ribonucleic acid (mRNA) that code for a virus antigen that is often found on the surface of the cell. These two strategies while potentially effective do not guarantee immunity to all.

Alternative immunization approaches include the use of monoclonal or polyclonal antibody treatments. One particular challenge with this approach is the relatively short half-lives of antibodies. For example, immunoglobulin class G (IgG) have half-lives on the order of 20 days. There are methods to modify the immunoglobulin to increase the half-life of the antibodies by a factor of 2. Additionally, immunoglobulins have been engineered in a variety of ways to enhance their efficacy. As an example they may be engineered to reduce receptor binding to the fragment crystallizable region (Fc) that allows them to be bound by engulfed by immune cells when the immunoglobulins are bound to an antigen. Such engulfing can result in antibody dependent enhancement of infection when antibodies lack high binding affinities and especially when antibodies decline in numbers or which can happen rather quickly given the relatively short half-lives. (See e.g., Iwasaki, A. & Yang, Y., 2020, Nat. Rev. Immunol. 20:339-341.)

Another challenge associated with this approach is that the antibodies are often developed in an animal such as a murine or non-human primate model and thus safety is not fully established in human. Thus, the time period between development and identification of the immunoglobulin and the potential to achieve FDA approval can take longer than what is ideal for a pandemic virus. Because pandemic viruses can have significant health costs and economic costs associated with them on the order of more than on thousand lives lost per day, unknown future complications for those infected and $billions per day in economic costs include job loss and time out of school reducing the time to FDA approval can have a significant safety and economic benefit for the entire country.

When individuals become infected with COVID, COVID-19 or other pandemic viruses the use of convalescent plasma derived from blood donations of those recovered from that virus may be administered to those that are hospitalized to aid in combating and recovering from the virus. However, there is an inherent shortage of convalescent plasma. Although, interestingly convalescent plasma received immediate designation as an investigational product even though it consists of a polyclonal mix of immunoglobulins some of which may elicit an immune response from the recipient. At the same time what is among the chief importance of convalescent plasma are the high affinity immunoglobulins for the viral antigen of interest. One drawback of the convalescent plasma is that some sources may not include antibodies of sufficiently high affinity for the virus of interest and therefore one donor source could be far more effective at combatting the virus than another. Nevertheless, that convalescent plasma is automatically considered an investigation product FDA is important and is a reflection of the fact that the FDA recognizes that nature has effectively become the investigational new drug (IND) enabling study and if immunoglobulins were developed in a human with a reasonably competent immune system any such B-cell source of immunoglobulins that would be unsafe because of too great an affinity for self antigens would not be activated by CD4+ helper T-cells. Ideally, all convalescent plasma sources would provide a large quantity of high affinity immunoglobulins for the virus of interest.

In addition convalescent plasma has many elements that can result in complications as a result of a plasma transfusion. Those risks include “(1) transfusion related acute lung injury; (2) transfusion associated circulatory overload, and (3) allergic/anaphylactic reactions.”, (See e.g., Pandey, S., & Vyas, G. N., 2012 Transfusion, 52, Supp1: 65S-79S.) What is important about this approach is that the antibodies developed in otherwise healthy humans that were infected with COVID is that the antibodies had to undergo a process of development known as affinity maturation that generally results in immunoglobulins with decreasingly smaller dissociation constants (K_{d}). In the case of COVID those antibodies are not always potent against the spike glycoprotein of COVID-19 and mutants.

The aim of a vaccine is to help humans develop adaptive immunity to a pathogen through exposure of the recipient to weakened forms of that pathogen or through exposure of the vaccine recipient to antigens that are typically presented on the surface of the pathogen. A challenge related to COVID is that there may be a large number of humans in the population that lack the ability to develop immunity to COVID through adaptive immune mechanisms. This may be due to immunosenescence, diabetes, a poorly regulated immune system, a limited repertoire of genetic elements that make up the V-regions of the adaptive immune system, poor regulation of B-cell development or due to other risk factors. Immunosenescence that results with increasing age also can result in higher pathogenicity of viruses due to associated changes in the aging lung. What is clear is that if at risk populations had the ability to express immunoglobulins with high affinity for the viral antigen or antigens of interest that did not rely on adaptive immune mechanisms they may be protected from the virus of interest such as COVID-19 and its mutant forms. For a review on immunosenescence and also the aging lung see e.g., Boe, D. M., et. al., 2017, Clinical and experimental immunology, 187:16-25. For a review of poor regulation of B-cell development see e.g., Vale, A. M., & Schroeder, H. W., Jr., 2010, The Journal of allergy and clinical immunology, 125:778-787. For a review of COVID-19 risk factors see the Centers for Disease Control website which is frequently updated.

### SUMMARY OF THE INVENTION

To address this concern it is proposed to take the safest and most potent immunoglobulins that can be identified in those that were infected by and recovered from COVID and infuse uninfected humans with a mix episomes each that encode for a potent immunoglobulin against COVID and collectively which encode for a polyclonal mix of immunoglobulins all based on at a minimum the V-regions “if not and preferentially the entire polypeptide sequence of the potent immunoglobulins” produced by an individual that recovered from COVID that is produced by a CD27+ IgG or IgA memory B-cell. Infusion of episomes will engender humans with the ability to express dimeric immunoglobulin class A subtypes 1 and 2 (dIgA1 and dIgA2)—potent for COVID-19 and its mutants—that becomes secretory immunoglobulin A (SIgA) as part of binding to polymeric immunoglobulin secreting receptor (pIgR) at the basal face of epithelial cells and entering the mucus of organs such as the upper respiratory tract and lungs. Additionally, the infusion of episomes will also engender humans with the ability to produce Immunoglobulin Class G (IgG) with emphasis on subtype 1 (IgG1) and IgG3 in addition to IgA1 and IgA2. This infusion of episomes engenders humans with two lines of defense against COVID both mucosal immunity and hematological immunity. Similarly, this approach could be used for any pandemic and even non-pandemic virus

The value of mucosal immunity against COVID is that it is possible to stop COVID before it breaches the epithelial barrier. If COVID is neutralized in the mucus and then this should eliminate the possibility of cytokine storm, Kawasaki Disease, auto immunity, organ damage and blood clots. Humans would be safe from these effects of COVID because in order for these complications to occur COVID must infect epithelial cells. Although, if COVID-19 or its mutants did manage to enter the bloodstream a second line of defense, episomes encoding for Immunoglobulin Class G (IgG) and immunoglobulins class A (IgA) would neutralize COVID-19, likely preventing adverse health effects.

Central towards this end and disclosed in this document is an effective and efficient process to identify isolate cells expressing the immunoglobulins of interest from a human blood or plasma source and to determine the DNA and/or polypeptide sequence of the immunoglobulins expressed by those cells. (For a basic scheme see FIG. 5)

### BRIEF DECRIPTION OF THE DRAWINGS

FIG. 1 depicts the relative location of the constant region genes for the immunoglobulin heavy chain on human DNA chromosome 14 as a function of immunoglobulin class. C_{γ} is the constant region DNA encoding for Immunoglobulin Class G. There are 4 subclasses of immunoglobulin Class G including C_{γ1}, C_{γ2}, C_{γ3} and C_{γ4} and their relative locations on DNA are shown. C_{α} is the constant region DNA encoding for Immunoglobulin Class A. There are 2 subclasses of immunoglobulin Class A including C_{α1} and C_{α2} and their relative locations on DNA are shown.

FIG. 2 depicts the mechanism by which Dimeric immunoglobulin classes A1 (dIgA1) and A2 (dIgA2) crosses the epithelium from the basal face—or face that is exposed to the tissue—of the epithelium by first binding to Polymeric Immunoglobulin Secreting Receptor (SIgR) that includes secretory component where it undergoes endocytosis into the epithelium and is transcytosed across the epithelium to the apical face—of face that is exposed to the mucus in the lumen—where upon exocytosis into the lumen of the organ of interest secretory component is transferred from SIgR to dIgA which is now referred to as secretory immunoglobulin class A or SIgA which refers to the fact that dIgA now has secretory component bound to it.

FIG. 3 depicts dimeric immunoglobulin class A (dIgA) which can exist for both subclasses of immunoglobulin class A subclasses dIgA1 and dIgA2. J-chain is also depicted.

FIG. 4 depicts SIgA1 and disulfide bonds between J-chain's cysteine 14 and 68 and IgA1 heavy chains terminal cysteine peptide. Also, shown in the disulfide bond between secretory component cysteine 502 and immunoglobulin class A1 heavy chain cysteine 311. The antibody-binding fragment (Fab) refers to the entire light chain and the Variable Heavy (V_{H}) domain and the Constant Heavy Domain 1 (C_{H1}). The 5 domains D1, D2, D3, D4 and D5 of secretory component are also depicted in this figure. The structure of SIgA1 is also represented as a crystal structure as a superposition of the 50 best-fit models. This model lightly illustrates that secretory components presence relative to the hinge regions of IgA1 make it more difficult for proteases to access the large hinge regions in SIgA1 thereby allowing SIgA1 to better resist proteolysis over IgA1 in mucosal environments including the upper respiratory tract, lungs and stomach.

FIG. 5 depicts the basic method to identify and express a polyclonal mix of immunoglobulins through a mixture of episomes delivered by an adeno-associated virus or lentivirus based delivery system. As a first step the blood or plasma of a human infected with COVID, COVID-19 or other pandemic or non-pandemic virus is collected the blood will then subjected to blood fractionation where the buffy coat or the thin layer that contains the leukocytes and platelets is extracted and the memory B-cells are extracted (step not shown). The IgA and IgG memory B-cells are subsequently extracted. Those IgG and IgA memory B-cells are then subject to a competitive binding assay with a virus antigen such as the COVID spike glycoprotein and separated through flow cytometry potentially using a fluorescence-activated cell sorting (FACS) cell separation method (details not shown) or a magnetic pull down separation using biotinylated magnetic beads bound to the antigen of interest where a subsequent competitive binding assay could be used with the identified memory B-cells. Isolated memory B-cells that are strongly binding to the antigen of interest would have their DNA sequenced with the DNA responsible for the V-Regions of the light and heavy chain immunoglobulins identified as well as the constant region DNA will also be identified. Vector constructs will be produced that encode for the identified immunoglobulins or V-regions as identified from the isolated memory B-cells with different possible sources for the constant region DNA genetic information including isotype switching and engineered constant region sequences to enhance function. Following a battery of tests that ensure high binding affinity and safety for the immunoglobulins vectors will be designed to be delivered to the cells of interest using an AAV, lentivirus or vesicle based delivery system.

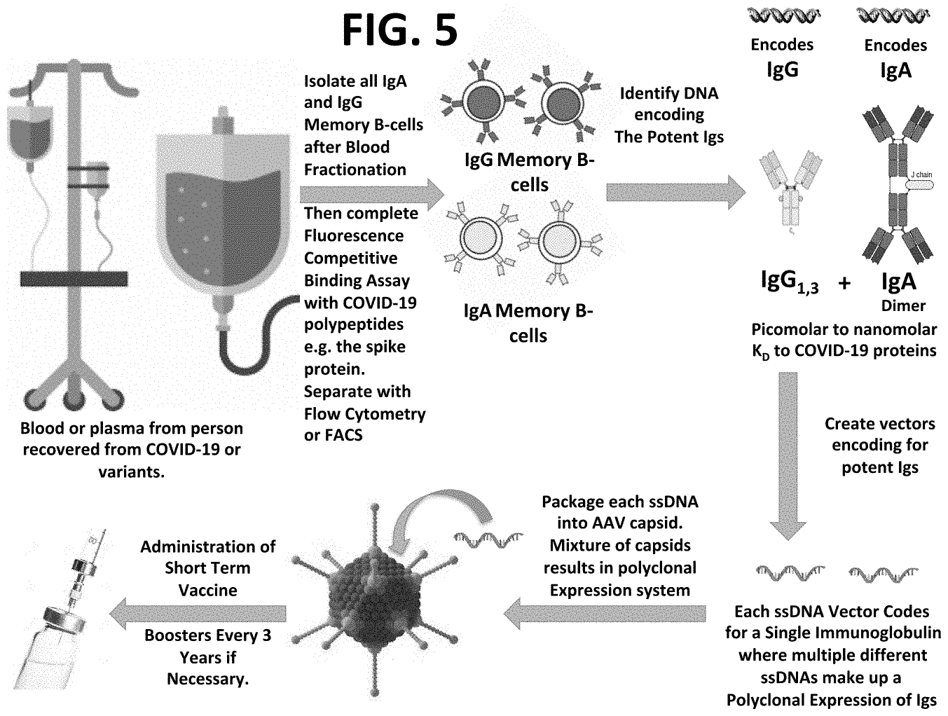

FIG. 6 depicts an AAV vector containing an inverted terminal repeat (ITR), a CMV promoter, a DNA sequence encoding IgHA1 or IgHA2 heavy chain immunoglobulin with the stop codon removed, DNA encoding a furin cleavage site, DNA encoding a 2A self cleaving peptide, a DNA sequence encoding IgLκ or IgLλ light chain with stop codon, an IRES, DNA encoding for the J-chain polypeptide necessary for the formation of dimeric Immunoglobulin A (dIgA), a polyadenylation element and finally an inverted terminal repeat.

FIG. 7 is similar to FIG. 15 and only differs by the relative location of the DNA encoding for IgH and IgL. FIG. 16 depicts an AAV vector containing an inverted terminal repeat (ITR), a CMV promoter, a DNA sequence encoding IgLκ or IgLλ light chain with the stop codon removed, DNA encoding a furin cleavage site, DNA encoding a 2A self cleaving peptide, a DNA sequence encoding IgHA1 or IgHA2 heavy chain immunoglobulin with stop codon, an IRES, DNA encoding for the J-chain polypeptide necessary for the formation of dimeric Immunoglobulin A (dIgA), a polyadenylation element and finally an inverted terminal repeat.

FIG. 8 depicts an AAV vector containing an inverted terminal repeat (ITR), a CMV promoter, a DNA sequence encoding IgLκ or IgLλ light chain with the stop codon removed, DNA encoding a furin cleavage site, DNA encoding a 2A self cleaving peptide, a DNA sequence encoding IgHA1 or IgHA2 heavy chain immunoglobulin with stop codon removed, DNA encoding a furin cleavage site, DNA encoding a 2A self cleaving peptide, DNA encoding for the J-chain polypeptide necessary for the formation of dimeric Immunoglobulin A (dIgA) with the stop codon removed, DNA encoding a furin cleavage site, DNA encoding a 2A self cleaving peptide, DNA encoding for a MZB1 protein with stop codon, a polyadenylation element and finally an inverted terminal repeat. The relative locations of the light chain immunoglobulin, heavy chain immunoglobulin and J-chain gene elements can be rearranged in the construct as it is presumed there is no materially different outcome with such rearrangements.

FIG. 9 depicts an AAV vector containing an inverted terminal repeat (ITR), a CMV promoter, a DNA sequence encoding IgLκ or IgLλ light chain with the stop codon removed, DNA encoding a furin cleavage site, DNA encoding a 2A self cleaving peptide, a DNA sequence encoding IgHAl or IgHA2 heavy chain immunoglobulin with stop codon removed, DNA encoding a furin cleavage site, DNA encoding a 2A self cleaving peptide, DNA encoding for the J-chain polypeptide necessary for the formation of dimeric Immunoglobulin A (dIgA) with the stop codon removed, DNA encoding a furin cleavage site, DNA encoding a 2A self cleaving peptide, DNA encoding for a MZB1 protein with stop codon, Woodchuck hepatitis virus posttranscriptional regulatory element (WPRE) (For an example see SEQ ID NO. 3), a polyadenylation element and finally an inverted terminal repeat. The relative locations of the light chain immunoglobulin, heavy chain immunoglobulin and J-chain gene elements can be rearranged in the construct as it is presumed there is no materially different outcome with such rearrangements.

FIG. 10 depicts an AAV vector containing an inverted terminal repeat (ITR), a CMV promoter, a DNA sequence encoding IgLκ or IgLλ light chain with the stop codon removed, DNA encoding a furin cleavage site, DNA encoding a 2A self cleaving peptide, a DNA sequence encoding IgHA1 or IgHA2 heavy chain immunoglobulin with stop codon removed, DNA encoding a furin cleavage site, DNA encoding a 2A self cleaving peptide, DNA encoding for the J-chain polypeptide necessary for the formation of dimeric Immunoglobulin A (dIgA) with the stop codon removed, DNA encoding a furin cleavage site, DNA encoding a 2A self cleaving peptide, DNA encoding for a MZB1 protein with stop codon, a polyadenylation element and finally an inverted terminal repeat. The relative locations of the light chain immunoglobulin, heavy chain immunoglobulin, J-chain and MZB1 gene elements can be rearranged in the construct as it is presumed there is no materially different outcome with such rearrangements.

FIG. 11 depicts an replication-deficient Lentiviral vector containing a 5′ deleted Long Terminal Repeat left orientation (dLTR) and a 3′-deleted Longer Terminal Repeat right orientation (dLTR) As an example the vector depicts 4 transgenes for expression of dimeric immunoglobulin A (dIgA) including the immunoglobulin heavy chain for isotype A (IgA), the immunoglobulin light chain (IgL) that may be kappa or lambda. The J-chain that may or may not include the signaling peptide and Marginal Zone B1 Cell Specific Protein (MZB1) that may or may not be included in the vector. Including in the vector but not necessarily shown (e.g. psi is omitted as a matter of convention) are all the elements including the central polypurine tract-central termination sequence (cPPT/CTS) needed for encapsidation of the viral genome. Also included are typical promoters e.g. CMV, an internal ribosome entry site IRES that could be substituted for with a polyA sequence (e.g. SV40 or BGH) and EF1-alpha promoter, a WPRE and polyA (e.g. SV40 or BGH) that is inclusive of all the elements necessary for transgene expression of the episome in the host cell.

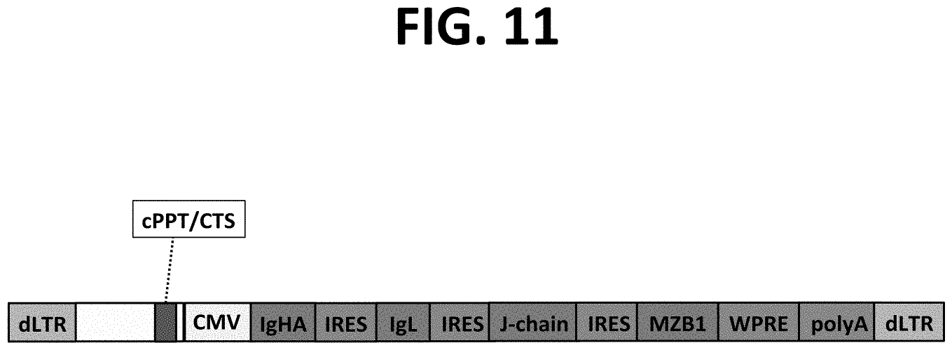

### DETAILED DESCRIPTION OF THE INVENTION

This present invention and proposed vaccination/immunization is designed to achieve safety and efficacy by embracing the most potent immunity developed in healthy humans exposed to COVID, COVID-19 its mutants or other pandemic and non-pandemic viruses. The invention describes the method to identify the DNA sequence and/or polypeptide sequence of high affinity immunoglobulins expressed in individuals that were infected with the virus of interest. The invention further describes the method to design vectors expressing those immunoglobulins and also dimeric immunoglobulins class A (DIgA) necessary for mucosal immunity that encode at a minimum the V-regions if not the entire polypeptide sequence of the potent immunoglobulins identified from the memory B-cells isolated from persons that were infected with the virus of interest. Delivery systems are also described which include AAV, lentivirus and vesicle based delivery systems.

Blood will be collected from individuals that were infected with the virus of interest such as COVID. Memory B-cells will be collected from the buffy coat layer (layer that contains white blood cells) of fractionated blood. The Immunoglobulin class A (IgA) and class G (IgG) memory B-cells will be subsequently isolated. Memory B-cells ells are the only B-cells that have both undergone some affinity maturation and bear cell surface immunoglobulins in any appreciable quantity that is required for investigation. Thus, the process of separating cells—that have undergone affinity maturation—based on the affinity of their immunoglobulins for an antigen of interest can only reliably take place with memory B-cells. Otherwise methods that consider every single B-cell would have to be utilized such a process in not only inefficient but cumbersome and likely to not result in the identification of B-cells expressing potent immunoglobulins because the number of cells that would need to be considered would increase by several orders of magnitude and therefore would be an unrealistic and unreliable method to identify high affinity immunoglobulins developed in humans that were infected with the virus of interest.

The genetic information that encodes for that immunity or the polypeptide sequence that results in that immunity will be determined, evaluated for safety and the incorporated into an episomal expression vector for expression in muscle cells, B-cells and other potential cells. Those episomes would be delivered via an adeno-associated-virus (AAV) vehicle or lentivirus a mixture of single-stranded or double stranded DNA episomes respectively that encodes for at a minimum immunoglobulins with V regions that exactly match those V Regions if not the entire polypeptide sequence of potent immunoglobulins for COVID, COVID-19, its mutants or other pandemic or non-pandemic viruses expressed in healthy humans that were infected by COVID, COVID-19 its mutants or other pandemic or non-pandemic viruses especially respiratory viruses. The polyclonal mixture of immunoglobulins will include both immunoglobulin class G (IgG), immunoglobulin class A (IgA) and dIgA. DIgA, which is converted to secretory IgA (SIgA) as part of being transported across the epithelium into the lumen of the upper respiratory tract, lungs and intestinal tract is the primary immunoglobulin responsible for mucosal immunity will also be encoded for in the polyclonal mix. Thus, it is through an AAV delivered single-stranded DNA episomes that collectively code for a polyclonal mixture of potent immunoglobulins that we will achieve immunity to COVID, COVID-19 and its mutants or other pandemic or non-pandemic virus for those who receive the vaccine/immunization.

Thus, the episomes encoding for the immunoglobulins of interest will produce full or even abbreviated length immunoglobulins that lack the Fc regions that can contribute to antibody dependent enhancement of infection. (See e.g., Iwasaki, A. & Yang, Y., 2020, Nat. Rev. Immunol. 20:339-341.)

The various compositions and methods of the invention are described below. Although particular compositions and methods are exemplified herein, it is understood that any of a number of alternative compositions and methods are applicable and suitable for use in practicing the invention. It is also understood that any evaluation of the immunoglobulin expression constructs for the vectors specified in this invention can be implemented using methods standard in the art. New vector constructs are also proposed to enable the expression of dimeric immunoglobulin A (dIgA) which becomes secretory immunoglobulin A (SIgA) as part of crossing an epithelial cell from the basal to apical face. Additionally, it is understood that there may be a number of methods and assay modifications that can be used to isolated memory B-cells based on relative affinity for antigens of interest by a flow cytometry or Fluorescence activated cell-sorting (FACS) technique. This should not dilute the value of any particular cited method. As the value of this invention lies in part by the efficiency that memory B-cells with immunoglobulins potent for a virus of interest may be identified from persons that were previously infected by that virus. Additionally, the value is further justified by the incorporation of genetic information to expression all or part of those high affinity immunoglobulins in a vector construct. Thus, it is not just the individual of the elements of the claims where the value is derived but their collective functioning together that results in a final product that can mitigate the effects on society brought about by particular pandemic viruses such as COVID.

The methods of this invention will utilize unless specified otherwise modern techniques of cell molecular biology, chemical biology, microbiology, biochemistry and immunology. Many of such techniques are explained substantially in the literature and are well understood to those skilled in the art.

1. Definitions

U Unless stated otherwise all terms used herein have the same meaning that they would to one skilled in the science, art and practice that is utilized in the present invention which include terms from methods in molecular biology, immunology, microbiology, chemical biology, recombinant DNA technology, biochemistry and virology. These terms are well within the knowledge of those with in depth knowledge or skill of the art5

The term “conserved polypeptide sequence” as used herein refers to polypeptide sequences that have remained evolutionally conserved possibly due to functional constraints. This bears relevance in the innate immune system, which relies on pattern recognition of conserved polypeptide sequences to recognize antigens of pathogens0

The term “affinity maturation” as used herein refers to the maturation of an immature B-cell through both isotype switching and somatic hyper mutation and a memory B-cell through somatic hyper mutation that occurs through CD4+ helper T-cell activation of B-cells that have engulfed and degraded an antigen.

The term “isotype switching” as used herein refers the changing of an immature IgD or IgM B-cell to an IgG, IgA or IgE B-cell as part of affinity maturation. Isotype switching exclusive of somatic hyper mutation may enhance the specificity of an immunoglobulin for its antigen.

The term “immunocompetent” as used herein refers to individuals that have the ability to produce and further develop an immune response following exposure to an antigen.

The term “pseudotyped” as used herein is the process of producing viruses or viral vectors in combination with foreign viral envelope proteins. A virus that is said to be pseudotyped is also referred to as a pseudovirus.

The term “daughter” cell as used herein refers to a memory B-cell of plasma secreting cell that results from a differentiated memory B-cell during affinity maturation.

The term non-viral vector may refer either to a virus or viral particle capable of functioning as an episome in the nucleus of host a cell but cannot integrate in the host genomic DNA. Non-viral vectors and transfer plasmids contain structural and/or functional genetic elements that are primarily derived from a virus.

The term “retroviral vector” refers to a viral vector or plasmid containing structural and functional genetic elements, or portions there of, that are primarily derived from a retrovirus.

The term “lentiviral vector” refers to a vector or plasmid containing structural and functional genetic elements, or portions thereof, including LTRs or dLTRs that are primarily derived from a lentivirus.

The terms “lentiviral vector,” “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles of the invention and are present in DNA form in the DNA plasmids of the invention.

The terms “integration-deficient lentiviral vector,” may be used to refer to lentiviral non-viral vectors that are capable of functioning in the host cell as an episome.

2. Isolation, Identification and Characterization of a Natural Human Source of Immunoglobulins with High Affinity for the Pathogenic Antigens of Interest.

The affinity of an immunoglobulin for its antigen which is inversely related to the dissociation constant (K_{d}) or the is an important determinant in one's ability to fight viruses that evade the innate immune system's ability to detect conserved polypeptide sequences of some foreign bodies that lack the conserved sequences the innate immune system can detect. With an increased duration of exposure to viruses the adaptive immune system can increases its affinity for viral antigens through a process of affinity maturation. When a B-cell becomes immature it typically starts out as an immunoglobulin class M (IgM) or immunoglobulin class D (IgD) B-cell that also bears a cell surface immunoglobulin class M receptor. Part of the B-cell development process consists of the early pro-B cell and pre-B cell gene element rearrangements to ensure productive matches and also the immature B-cell detects in the bone marrow for self-antigens. When the affinity for a self-antigen is above a specific threshold the immature B-cell can undergo apoptosis. Immature IgM and IgD B-cells have heightened sensitivity to antigen-induced apoptosis. This is an important safeguard to ensure that only those B-cells that do not have high reactively to self can undergo a further process of affinity maturation. (See e.g., Melamed, D., 1998, Cell. 92:173-182) However, about 20% of immature B-cells have some reactivity to self-antigens (See e.g., Wardemann, H., et. al., 2003, Science 301:1374-1377). As without this we would not be able to sufficiently generate an adaptive immune response to a broad range of antigens. Immature B-cells undergo further differentiation into mature B-cells or naive B-cells that continue to bear cell surface immunoglobulins if IgM or IgD.

During the affinity maturation process cell surface immunoglobulin bearing B-cells can undergo a process of somatic mutation as well as isotype switching to potentially increase the immunoglobulins affinity for pathogenic antigens. Both these process can potentially result in B-cells with reactivity to self-antigens. Naive B-cells give rise to both lymphoblasts that secrete antibodies and memory B-cells upon activation from a pathogenic antigen and the activation of CD4+ helper T-cells. Helper T-cells ensure that the antigen bound to and degraded by the memory B-cell are not self-antigens. When a B-cell is activated by a CD4+ helper T-cell it differentiates and affinity matures into antibody secreting cells that do not bear a cell surface immunoglobulin or a memory B-cell that does bear a cell surface immunoglobulin. Through, repeated process of memory B-cell activation by helper T-cells daughter memory B-cells or daughter plasma secreting cells can develop a greater affinity for the antigen of interest. (See e.g., Ziegner, et. al., 1994, Eur. J. Immunol. 24:2393-2400; Shlomchik, M. J., et. al., 1990, Prog. in Immunol. Proc. 7:415-423; Berek, C., & Milstein, C., 1987, Immunol Rev. 96:23-41). It is through this process that affinity matured memory B-cells can for the most part generally be considered to express immunoglobulins that have high affinity for an antigen of interest and low affinity for self-antigens. Although, there are exceptions as during the course of any somatic mutation or isotype switch process that results in memory B-cells the possibility exists that memory B-cells can have a higher affinity for self-antigens. There is a panel of assays and additional assays that are used to determine if immunoglobulins have reactivity to self-antigens. (See e.g., Tiller, T., et. al. 2007, Immunity, 26:205-213; Also see e.g., Fsuiji, M., et. al. 2006, The Journal of Experimental Medicine 203:393-400, 2006.; Wardemann, H., et. al., 2003, Science 30:1374-1377)

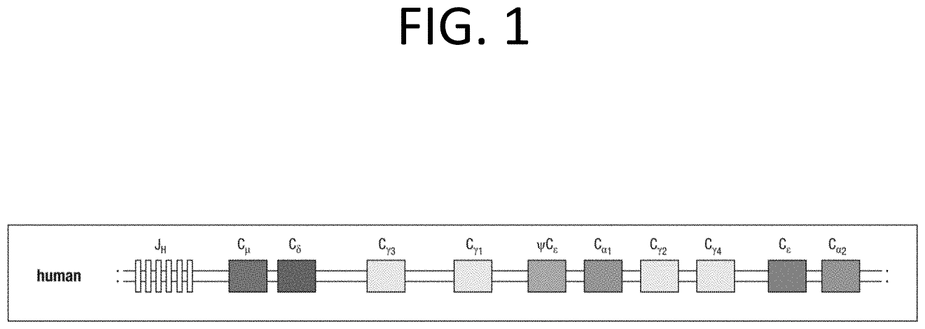

This element of the present invention now provides a method to identify IgG and IgA immunoglobulins with high binding affinity to the antigen of interest that are naturally produced in immunocompetent individuals that have been infected with the virus of interest such as COVID, COVID-19 and mutants and other pandemic or non-pandemic viruses or pathogens. Vectors may be created that encode for those immunoglobulins to be evaluated. Additionally, the V-region DNA of the heavy (IgH) and light chain (IgL) of the immunoglobulins may be incorporated into a vector construct that could use either the constant region DNA identified in the B-cell expressing (See FIG. 1) the identified immunoglobulin of interest or another source of genetic information may replace all or part of the constant region DNA that may also include isotype switching of constant region genetic information, mixes of two constant regions from two isotypes that may be accomplished with combinations of Fab and natural or engineered Fc domains or F(ab′)₂ and natural or engineered pFc′ domains. Central towards this end is the production of dIgA1 to enable mucosal immunity against the virus of interest especially with respiratory transmission is common so that a large variety of complications associated with infection and can be avoided and individuals can have sustained short term immunity to the pathogen of interest.

A human (ideally between 21 and 55 years old) that was infected with the pathogen of interest such as COVID, COVID-19 and mutants or other pandemic or non-pandemic viruses or pathogens has their blood drawn. Half that blood or convalescent plasma may be used to treat another human with COVID19 to aid recovery. Although, this transfusion step is not necessarily required in order for a blood sample to be considered but could be helpful. The blood sample will subsequently be subjected to blood fractionation method such as density gradient centrifugation. The buffy coat layer, which contains the peripheral blood mononuclear cells (PBMCs) that includes memory B-cells is collected.

It may be beneficial to identify families where one family member is having a difficult time developing immunity from COVID-19 and the other is not developing immunity. This would result in repeated exposure of one immune partner from the other partner that does not have immunity. As a result the immune partner will have undergone multiple rounds of affinity maturation and may have strongly binding antibodies with K_{d} values ranging from sensitivities of 10⁻¹⁰ to 10⁻⁸ or the picomolar to nanomolar sensitivity range (K_{off}/Kₒₙ=K_{d}). In a similar situation a medial staff could have contracted COVID-19 and got sufficiently sick recovered in the hospital and is now able to treat humans and not get infected again.

If there is a resulting blood or plasma transfusion from a particular donor and the donation results in recovery of the recipient the other half of the blood or plasma would have the CD27+ IgG+ memory B-cells isolated. The idea behind requiring the recovery is that if the antibodies of the donor have strong binding affinity then they are more likely to neutralize the COVID-19 in the recipient. However, a blood or plasma transfusion to another human ill with COVID-19 is not necessarily required for an individual to be identified to have potent antibodies for COVID-19 or other virus of interest.

One method that could be used to isolate CD27+ IgG+ memory B cells would be with EasySep™ Human IgG+ Memory B Cell Isolation Kit¹. This kit can be purchased from STEMCELL Technologies in Cambridge, Mass. This kit also includes an option to separate out CD27+ IgA+ memory B-cells. In a similar sense all of the memory B-cells could also be isolated and analyzed together. The separation of different memory B-cell isotypes is achieved through the use of immunoglobulins specific for the heavy chain Fragment crystallizable (Fc) constant regions of a specific memory B-cell isotype. ¹Website: https://www.stemcell.com/easysep-human-igg-memory-b-cell-isolation-kit.html#section-data-and-publications

Using the STEMCELL Technologies method primary human CD27+ memory B-cells can be isolated by immunomagnetic bead isolation followed by a depletion cocktail to deplete Human IgM/IgD/IgA B-cell Depletion Cocktail leaving the CD27+ IgG memory B-cells as a pure sample. Also available is a solution to deplete Human IgA B-cells. Thus, both IgG and IgA memory B-cells will be collected. (Catalog #17868, STEMCELL; Website: https://www.stemcell.com/easysep-human-igg-memory-b-cell-isolation-kit.html#section-data-and-publications) Patient PBMCs may be stored frozen and thawed before use. (For an alternative method to isolate CD27+ B-cells see e.g., Tiller, T., et. al. 2007, Immunity, 26:205-213 and references cited therein)

It is difficult to perfectly separate the IgG memory B-cells by their subclasses e.g. IgG1, IgG2, IgG3 etc. IgG1+ memory B-cells generally make up 60%-70% of IgG memory B-cells in healthy donor serum. IgG2+ memory B-cells make up about 25% of IgG memory B-cells and IgG3+ memory B-cells make up about 9% of IgG memory B-cells. This difficulty in separation suggests that the all IgG subclass have heavy chain constant regions that are topographically similar. (See e.g., Tiller, T., et. al. 2007, Immunity, 26:205-213) However, this is not important because downstream steps will address these differences accordingly.

IgG memory B-Cells or IgA memory B-cells will then be separated by relative binding affinity through a flow cytometry technique such as fluorescence activated cell sorting (FACS). The flow cytometry analysis of the relative binding affinities of memory B-cells will be dependent on labeling antigen such as spike protein with a fluorescent tag to allow detection of spike protein bound B-cells by the FACS. There are an extensive number of well-established methods to determine the relative binding affinities of cell surface receptors for the antigen of interest. As an example, one may use an Angiotensin Converting Enzyme 2 (ACE2) peptide in competition with memory B-cells to bind to the ACE2 receptor-binding domain (RBD) of the spike glycoprotein found on COVID-19 virions. The K_{d} of the ACE2 RBD with the spike glycoprotein is 15.9 nM. Assays may be designed to determine if memory B-cells binding affinities greater that of ACE2 for the spike glycoprotein RBD. There are well-established methods for approximate quantification using methodology, known to those of skill in the art. This would represent an important first step in determining which memory B-cells cell surface immunoglobulins have a high binding affinity for viral antigens. (See e.g., Weiss, G. E., et. al., 2012, Journal of immunological methods, 375:68-74.; Hunter, S. A., & Cochran, J. R., 2016, Methods Enzymol. 580:21-44.; Li, P., Selvaraj, P., & Zhu, C., 1999, Biophysical journal, 77:3394-3406.; Amanna, I. J., & Slifka, M. K., 2006, Journal of immunological methods, 317:175-185)

Immunoglobulin DNA sequences of the memory B-cells can be obtained from single cell real time polymerase chain reaction (RT-PCR). Alternatively, an optofluidics platform could obtain immunoglobulin sequences on the order of several days through the execution of nested PCR on single antigen-binding memory B-cells after single cell sorting. These are well-established methods known to those of skill in the art and also available from a variety of commercial sources with standardized protocols. Further, such a method was previously established to allow for efficient isolation and identification of neutralizing monoclonal antibodies (mAbs) in different infectious diseases such as HIV. (See e.g., Setliff, I., et. al., 2019, Cell, 179:1636-1646)

Alternatively, memory B-cells specific to the antigen can be subjected to an antigen isolation assay where antigen biotinylated to magnetic beads is competed for between memory B-cell surface immunoglobulins such as IgG+ or IgA+ of different memory B-cells. Magnetic pull downs will only capture those memory B-cells with the greatest relative binding affinity for the antigen of interest. B-cells can also compete against a protein of interest that is present in the solution with known binding affinity to the biotinylated antigen. (See e.g., https://www.sinobiological.com/recombinant-proteins/2019-ncov-cov-spike-40592-v08h-b) The assay can be modified by adjusting the ratio of antigens and competing binding proteins (if one is used) to obtain a relatively small number of memory B-cells. These are well-established methods known to those of skill in the art and also available from a variety of commercial sources with standardized protocols. However, for any particular antigen assays may have to be developed using well-established methods known to those of skill in the art. If magnetic beads are used what would result are a cohort of memory B-cells that could then be separated with a subsequent competitive binding flow cytometry assay such as FACS as described herein.

The isolated memory B-cells can subsequently be stimulated to differentiate into antibody secreting cells that secrete monoclonal antibodies without somatic mutation occurring during the differentiation. The secreted antibodies can be evaluated for binding affinity using B-cell ELLISPOT. (See e.g., Weiss, G. E., et. al., 2012, Journal of immunological methods, 375:68-74) Alternatively, an Enzyme-Linked Immunosorbent Assays (ELISA) can be used to evaluate the secreted antibodies to estimate binding affinity for a specific antigen. Such approaches are well understood by those with skill in the art.

The K_{d} threshold for determining which memory B-cells or their in vitro generated and un-mutated daughter cells will be selected for further evaluation will be based on a number of factors including targeting a specific number of high affinity memory B-cells and achieving a mix of IgG1, IgG3, IgA1 and IgA2 memory B-cells each that meet a K_{d} criteria. Preferentially, the K_{d} criteria for memory B-cell selection would be in the single digit picomolar range. Such dissociation constants can be measured by surface plasmon resonance for the monoclonal antibody. Alternatively or additionally a neutralization titer (NT₅₀) inhibitory dose may be determined to assess relative binding affinities of immunoglobulins for an antigen of interest. The NT₅₀ is defined as neutralization titers [50% inhibitory dose (ID50) or the 50% inhibitory concentration (IC50)] that is defined as the reciprocal of the serologic reagent dilution (or concentration for purified reagents) that caused a 50% reduction in relative luminescence (RLU) compared to virus control wells after subtraction of background RLUs. As an example an input virus luminescence signal such as mNeonGreen, luciferase or mCherry reporter signal are often used. Neutralization rates are calculated as 1−[(RLU signal for virus plus plasma)/(RLU signal for virus only)]. Corresponding dose-response curves may be analyzed by nonlinear regression. The plasma concentration at which the neutralization rate equals 0.5 is given as the half-maximal neutralization concentration, and the reciprocal value is the NT₅₀. (See e.g., Antonio, E., et. al., 2020 bioRxiv, 2020.05.21; Sarzotti-Kelsoe, M., et. al., 2014, Journal of immunological methods, 409:147-160)

The DNA sequences that codes for the immunoglobulins expressed by the memory B-cells may be determined as described earlier. (E.g. Immunoglobulin DNA sequences of the memory B-cells can be obtained from single cell real time polymerase chain reaction (RT-PCR). Alternatively, an optofluidics platform such as Beacon could obtain immunoglobulin sequences on the order of several days through the execution of nested PCR on single antigen-binding memory B-cells after single cell sorting. Such a method was previously established to allow for efficient isolation and identification of neutralizing monoclonal antibodies (mAbs) in different infectious diseases such as HIV. (See e.g., Setliff, I., et. al., 2019, Cell,179:1636-1646)

Discussed earlier, the possibility exists that memory B-cells can have a high affinity for self. The probability of such a self-reactive immunoglobulin being produced is more likely during earlier rounds of affinity maturation. There is a panel of assays and additional assays that are used to determine if immunoglobulins have reactivity to self-antigens. There are well-established methods to carry out these panel assays known to those of skill in the art. (See e.g., Tiller, T., et. al. 2007, Immunity, 26:205-213; Also see e.g., Fsuiji, M., et. al. 2006, The Journal of Experimental Medicine 203:393-400, 2006.; Wardemann, H., et. al., 2003, Science 30:1374-1377). Another described global self-antigen assays can also be used to evaluate immunoglobulin for self-reactivity. (See 850 e.g., Vale, et. al., 2016, Front. Immunol. 7; Nobrega, A., et. al., 1993, Eur J Immunol., 23:2851-2859; Haury, M., et. al., 1997, Scand. J. Immunol. 39:79-87) The isolated memory B-cells can subsequently be stimulated to differentiate into antibody secreting cells that secrete monoclonal antibodies without somatic mutation occurring during the differentiation. (See e.g.,Weiss, G. E., et. al., 2012, Journal of immunological methods, 375:68-74)

3. Modification of Constant Regions of Natural or Engineered Sources of Human Derived Immunoglobulins with High Affinity for the Viral Antigens of Interest.

Those immunoglobulins that are high affinity for antigen and sufficiently low reactivity for self-antigens will have their DNA incorporated into an expression vector for further evaluation. That evaluation may include modifying the constant regions of the immunoglobulins using a variety of different sources of human genetic information. This approach may include using constant regions of different isotypes and engineering of the constant region that are detected by Fc receptor on macrophages and monocytes as an example. All such immunoglobulins may be evaluated in the battery of tests that include self-reactivity assays and binding assays as described in this document.

For IgG+ memory B-cells and regarding DNA sequencing: For the heavy chain sequence of the immunoglobulin the regions where the corresponding switch region recombination event resulting in DNA for different constant region genetic loci will be sequenced including Cγ_{1,2,3 or 4} IgG heavy chain regions. Additionally, the Cα₁ and Cα₂ conserved sequences that would result in the IgA1 or IgA2 isotype immunoglobulins may also be sequenced from the DNA of the IgG+ cells. Such methods to identify the relevant sequences at genetic loci responsible for immunoglobulin expression and also constant region genetic loci is part of established analytical techniques of DNA data from PCR that is within the aptitude to those of skill in the art.

Vectors may be created that encode for those immunoglobulins to be evaluated. Additionally, the V-region DNA of the heavy (IgH) and light chain (IgL) of the immunoglobulins will be incorporated into a vector construct that could use either the constant region DNA identified in the B-cell (See FIG. 1) expressing the identified immunoglobulin of interest or another source of genetic information may replace all or part of the constant region DNA that may also include isotype switching of constant region genetic information, mixes of two constant regions from two isotypes that may be accomplished with combinations of Fab and natural or engineered Fc domains or F(ab′)₂ and natural or engineered pFc′ domains. The resulting identified immunoglobulin genetic loci responsible for the memory B-cell binding affinity can further be incorporated into a gene expression vector that could combine an identified V-regions of both light and heavy chains with a constant region of another isotype or constant region that contains an engineered Fc region. Likely, the immunoglobulin light chain (IgL) constant region would be used as identified from the memory B-cell. If an CD27+ IgG1+ memory B-cell is identified to have potent binding to the antigen of interest not only will an expression vector encoding an IgG1 immunoglobulin be created but also expression vectors encoding each of IgG3, IgA1 and IgA2 coding for precisely the same V-region polypeptide—as in the identified IgG1+ CD27+ memory B-cell—will also be created to have a more comprehensive assessment. The rational for pair the V-region of one Ig isotype with that of another is that increased binding affinity can occur simply as a result of an isotype switch from IgM or IgD to IgG, IgA or IgE without changing the amino acid sequence of the V-region. Additionally, enhanced or reduced signal transfer from Fab to the Fragment crystallizable (Fc) region to the can occur simply as a result of isotype switching (See e.g., Pritsch, O., et al., 1996, J Clin Invest. 98:2235-2243.; Janda, A., et. al., 2016, Front Microbiol., 7:22).

4. Incorporation of the Immunoglobulin Genetic Information of IgG1, IgG3, IgA1, IgA2, dIgA1 Into Non-Viral Expression Vectors Intended for Different Delivery Vehicles such as Adeno-Associated Virus, Lentivirus or Vesicle based delivery systems to enable a polyclonal expression of immunoglobulins through episomal expression of DNA.

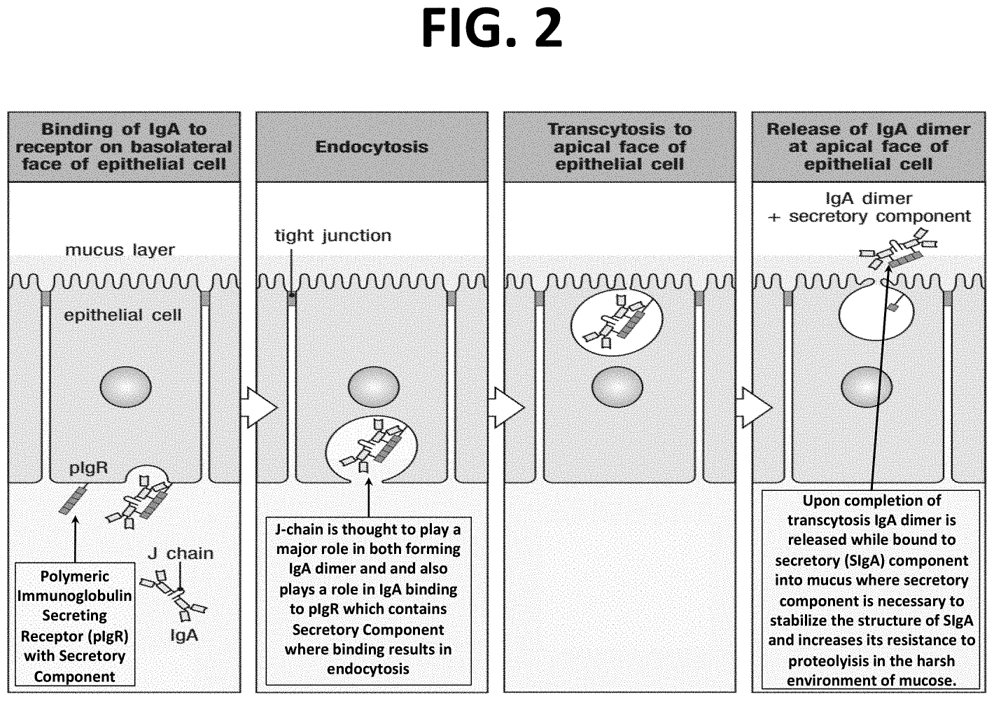

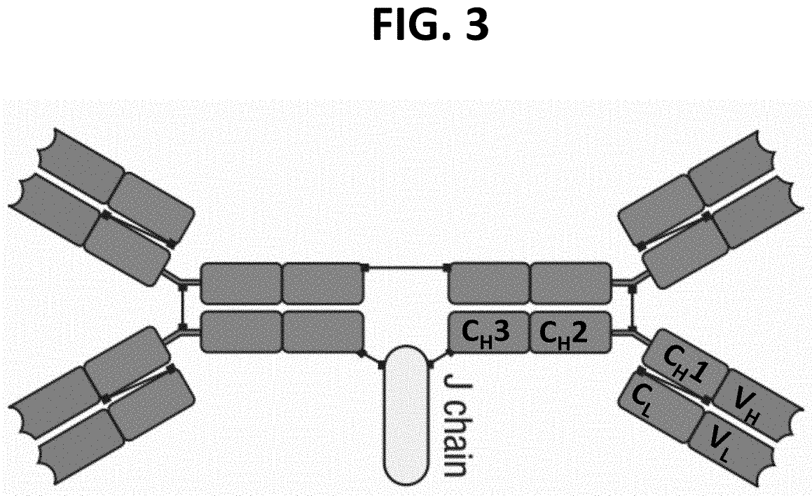

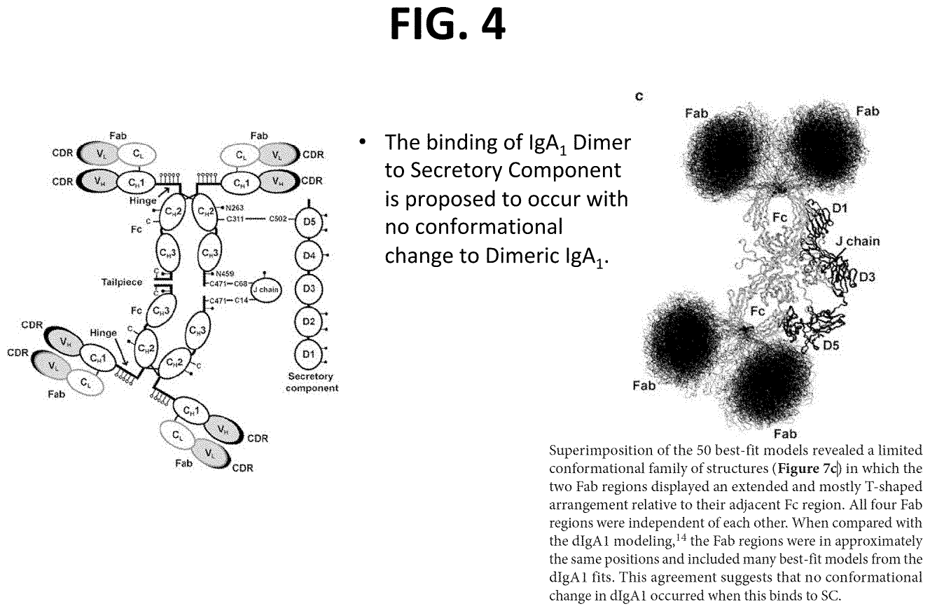

The immunoglobulins identified to both have high binding affinity (ideally single digit picomolar binding affinity) to antigens of interest and low reactivity to self-antigens are intended to be expressed from episomal DNA in the host cells. AAV, lentivirus or vesicle based delivery systems will be used to transport the vector constructs to their target cells. A mix of episomes each of which codes for a unique immunoglobulin and collectively which code for a polyclonal mix of immunoglobulins. As an example, there may be a total of 7 unique immunoglobulins that are coded for by episomes. Those immunoglobulins will include one or more of the following immunoglobulin classes: IgG1, IgG3, IgA1, IgA2 and dIgA1 (See FIG. 3). Mucosal immunity against the pathogen of interest is best achieved with SIgA1 that is a product of dIgA1 following transcytosis from the basal to apical face of an epithelial cell where dIgA1 forms a disulfide bond (See FIG. 4) with secretory component that is found on polymeric immunoglobulin secreting receptor (pIgR) as part of crossing the epithelial cell (See FIG. 2).

Immunoglobulins are dimers of heterodimeric proteins that consist of light and heavy chain proteins or moderate size linked together through a disulfide bond they can be difficult to express from vector constructs. Reports of the expression of immunoglobulin vector constructs have focused on the expression of immunoglobulins that are dimers of the heterodimers. That is the immunoglobulin consists of two identical heavy chains and two identical light chains linked together through disulfide bonds. One reason immunoglobulins can be difficult to express in vector constructs due to the limited capacity of the vectors. For example, the maximum capacity of an Adeno-Associated Virus (AAV) capsid is typically about 4.9 kilobases of single stranded DNA. Typically, to express two gene elements from a single vector construct there must be enough room for both DNA sequences, a promoter that includes a ribosome binding site, an intermediate promoter between the two gene elements that includes a ribosome binding site, a polyadenylation element (e.g. Semina Virus 40 polyadenylation element (SV40 polyA) (For an example see SEQ ID NO. 4) or Bovine Growth Hormone polyadenylation element (BGA polyA)) and potentially a post transcriptional regulatory element (e.g. Woodchuck Hepatitis Virus (WHP) Posttranscriptional Regulatory Element (WPRE)). In this context collectively, the sum of these elements approaches or exceeds the 4.9 kilobase capacity of the AAV vector. There have been a few reports and patents that address the challenge associated with obtaining sufficiently high expression of immunoglobulins that are dimers of heterodimers. There are two major strategies that may be used. The first is to express the light and heavy chain immunoglobulins as a single open reading frame. When two genes of the light and heavy chain immunoglobulins are expressed as a single open reading frame (that is the stop codon of the upstream gene is not encoded) a furin cleavage site (See Table 1) and a 2A self-cleaving peptide are used to ensure efficient yields and viable immunoglobulin production.

A furin cleavage site is used in conjunction with the 2A self-cleaving peptide to eliminate the large polypeptide residue that would otherwise be left at the N terminus residue on the upstream immunoglobulin chain after self-cleavage of the 2A self-cleaving peptide. Furin cleavage sites have a consensus sequence of RXKR—where X can be any amino acid—and can range from 4 to 6 peptides in length and are cleaved by furin or other proteases. It has been demonstrated that 2A residues are efficiently removed from the N-terminus of the upstream immunoglobulin chain by placing a furin cleavage site upstream of the 2A residue resulting in an immunoglobulin chain with a furin cleavage site on the N-terminus end following by the cleavage peptide N-terminus to the furin cleavage site. (See e.g., Fang, J., et al., 2005, Nat. Biotechnol., 23: 584-590.; Krysan, D. J., et. al., 1999, J Biol Chem. 274:23229-23234)

An alternative and earlier reported strategy utilized a dual-promoter rAAV vector to express immunoglobulins. CMV and SV40 small T-antigen intron was used as the promoter for the first transcriptional unit that was followed by the leader sequence and heavy chain sequence. The leader sequence is a 19 amino acid cleavable sequence that is expressed C-terminal on the heavy (Leader Sequence: MGWSCIFLFLLSVTVGVFS) and light chain (Leader Sequence MKLPVRLLVLMFWIPASSS) chain immunoglobulins and is necessary for efficient translocation into the endoplasmic reticulum. A Bovine Growth Hormone (BGH) polyadenylation (polyA) (For an example see SEQ ID NO. 5) site was used as the post-transcriptional regulatory element for the first transcriptional subunit. The second transcriptional unit utilized an abbreviated human elongation factor 1α (EF1-a) promoter that was modified to improve stability of DNA and RNA that also contained an intron 1117 (SEQ ID NO. 9) 5′Untranslated Region (5′UTR). An SV40 polyadenylation site was used as the post-transcriptional regulatory element for the second transcriptional subunit. (See e.g., Lewis, A. D., et. al., 2002, J Virol. 76:8769-8775; Huang, M. T., et. al., 1990, Molecular and cellular biology, 10:1805-1810; Wu, C., et. al., 2004, Molecular and cellular biology, 24:2789-2796).

Previously, these two considered vector constructs were used to express immunoglobulins that are dimers of hetero dimers in mammalian models. (See e.g., Fang, J., et al., 2005, Nat. Biotechnol., 23: 584-590; Lewis, A. D., et. al., 2002, J Virol. 76:8769-8775) However, these reports were not concerned with the expression of dimeric immunoglobulins (dIgA), which only exist for isotype A in human. Dimeric immunoglobulins are tetramers of heterodimers and requires J-chain for efficient formation and requires MZB1 to facilitate formation. No such reports or patents concerned with the expression of dIgA from a single AAV vector have been identified.

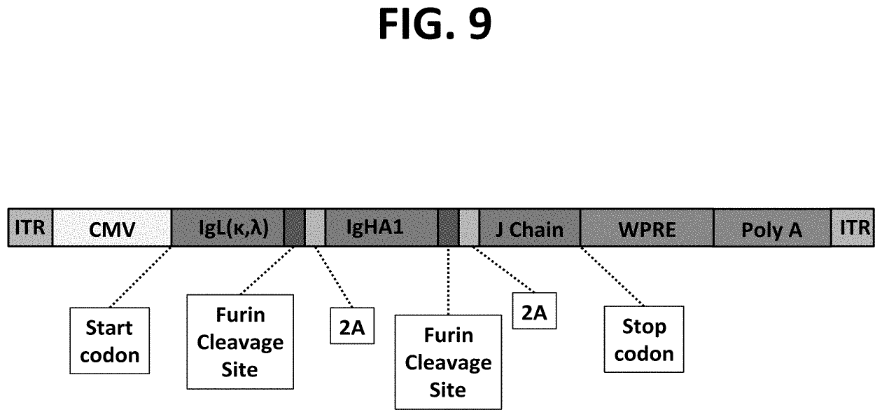

J-chain a 159 amino acid (SEQ ID NO. 11) protein with peptide signaling sequence MKNHLLFWGVLAVFIKAVHVKA that is cleaved to a 137 amino acid protein (SEQ ID NO. 11) is shown to be required for efficient production of dIgA. (See e.g., Lycke, N., et. al., 1999, J. Immunol.163:913-919; Sørensen, V., et. al., 1999, J. Immunol., 162:3448-3455; Schroeder, H., et. al., 2008, Fundamental Immunology (Book) 6^{th:}125-151; Castro, C. et. al., 2014, Journal of Immunology, 193:3248-3255; Koshland, M. E., 1985, Annu Rev Immunol. 3:425-453) Without J-chain there is a substantial reduction in the amount of dIgA produced by an organism such as mice. J-chain forms 2 disulfide bonds (See FIG. 9) with two IgA1s at the penultimate cysteine (C471) on the heavy chain tail of each IgA1 and C14 and C68 of J-chain. J-chain has been called the glue for the efficient formation of dIgA. (Bonner, A. et. al., 2009, Mucosal Immunol 2:74-84) J-chain has also been shown to be necessary for binding to secretory component on pIgR. (See e.g., Johansen, F. E., et. al. 2000, Scandinavian Journal of Immunology., 52:240-248) The partially buried ligand binding motifs of secretory component are thought to interact with J-chain of Dimeric IgA as part of inducing a conformational change to free domain 5 of secretory component on pIgR and form a disulfide bond between C502 of secretory component and C_{H}2 cysteine C311 of the immunoglobulin heavy chain of dIgA. (See e.g., Stadtmueller, B. M., et. al., 2016, Elife. 5:e10640).

A recent report has suggested that Marginal Zone B1 Cell Specific Protein (MZB1) (SEQ ID NO. 8) is also necessary for efficient formation of dIgA. For example MZB1 Double Knockout (MZB1^{-/-}) mice produce significantly lower quantities of dIgA than mice that have a functioning MZB1 gene. (See e.g., Xiong, E., et. al., 2019, Proceedings of the National Academy of Sciences of the United States of America, 116:13480-13489) Thus, to express dIgA is sufficient quantities it may required to co express MZB1 or to deliver the episome to a cell that sufficiently expresses MZB1 at large enough quantities to support the efficient formation of dIgA from an episome.

Without the availability of MZB1 and J-chain proteins dIgA would form in very small quantities and would fail to convert over to SIgA. MZB1 interacts with IgA through the α-heavy chain (aHC) tailpiece dependent on the penultimate cysteine residue (where two αHCs form a disulfide bond with J-chain cysteine C14 and C68) and prevents intracellular degradation of α-heavy chain—Light chain (αHC-LC) IgA Dimer complexes. MZB1 promotes J-chain binding to IgA and the secretion of dimeric IgA. If considering the delivery of episomes coding for dIgA1 to myocytes (muscle cells) where expression would occur from muscle cell nuclei MZB1 would to be encoded for in the vector. However, this instant patent also considers the expression of immunoglobulins from episomes that self-replicated in B-cells targeted by the delivery vehicle.

### Immunoglobulin Regions

Human immunoglobulins are complex proteins with different structural/functional regions. Fig. X depicts the different structural/functional regions of immunoglobulins. The domain of the immunoglobulin responsible for binding to the antigen of interest is referred to as the antigen-binding fragment (Fab) domain. The Fab domain is considered to be made up of the entire immunoglobulin light chain (IgL) that includes its V-region (V_{L}) domain and constant region domain (C_{L}) and the immunoglobulin heavy chain's V-region (V_{H}) domain and C_{H}1 domain. Researchers may also classify the Fab to include the hinge domain of the heavy chain and refer to it as F(ab′)₂ fragment. Both the Fab and F(ab′)₂ domains are considered as part of this patent filing. It is reasonably likely that one may be able to use the Fab or F(ab′)₂ fragments of an immunoglobulin with high affinity for an interested and shown in many cases that making some modification to the crystallizable fragment Fc (made up of all peptides that include the hinge, C_{H}2, and remaining domains N-terminal to C_{H}2) and pFc′ (made up of all peptides that include C_{H}2 and remaining domains N-terminal to C_{H}2) and show there is little if any change in affinity of the Fab and F(ab′)₂ for their antigen. One may modify or change—from another human coding source—such Fc and pFc′ fragments to also avoid the potential of an immune response against the Fc and pFc′ fragments. Alternatively, one may engineer the Fc and pFc′ fragments on IgG immunoglobulin to minimize their binding to the FC receptors such as FcγR in order to minimize antibody dependent enhancement of infection. (See e.g., Iwasaki, A. & Yang, Y., 2020, Nat. Rev. Immunol. 20:339-341). This instant patent considers such an approach. Additionally, this patent considers dIgA-COVID polymerization to be an effective potential means of virus neutralization in the respiratory mucosa. (see e.g., Terauchi, Y., et. al., 2018, Human vaccines & immunotherapeutics, 14:1351-1361.). During affinity maturation B-cells can alter their immunoglobulins by both somatic hyper mutation of the DNA coding for the V_{H} and V_{L} domains. Additionally, during affinity maturation B-cells can alter their immunoglobulins through isotype switching that is through switching the entire constant region polypeptide. This isotype switching can result in changing the Cμ constant region with any downstream constant region. All immunoglobulin class switch recombination events occur from IgM or IgD to IgG, IgA or IgE. For example, IgG to IgA class switching is not reported to be observed. (See e.g., Stavnezer, J., et al., 2014, Journal of immunology, 193:5370-5378.) However, class switching can see an observed increased in affinity. The rational for pair the V-region of one Ig isotype with that of another is that increased binding affinity can occur simply as a result of an isotype switch from IgM or IgD to IgG, IgA or IgE without changing the amino acid sequence of the V-region. Additionally, enhanced or reduced signal transfer from Fab to Fc can occur simply as a result of isotype switching (See e.g., Janda, A., et. al., 2016, Front Microbiol. 7:22).

Engineering Fc Region of Immunoglobulin to Prevent Antibody Mediated Viral Enhancement or to Improve Other Effector Functions

Engineering the Fc region of immunoglobulins can serve a large variety of functions including altering their half-life, to enhance complement dependent effector function, to enhance or reduce Fc receptor effector functions and to enhance binding affinity of immunoglobulins. The benefits of engineering the Fc regions to improve the antibody half-life or binding affinities are obvious. Enhancing complement dependent effector functions as enhancing the complement effector functions of an immunoglobulin leads to more rapid response by supporting immune cells and immune signaling proteins allowing the pathogen to be eliminated more rapidly. Complement generally comes into play once a pathogen breaches the epithelial barrier. However, should COVID breach the epithelial barrier the probably of cytokine storm is significantly higher. In some cases immunoglobulins may target pathogen cell surface proteins and prevent interactions between cell surface proteins and host receptors.

If may be beneficial to reduce or eliminate effector function to prevent cytokine secretions and the secretion of other pro-inflammatory agents that occurs with antibody mediation viral enhancement or antibody dependent enhancement (ADE) where it is possible for pathogen specific antibodies to promote pathology. Generally, this only occurs when antibody levels are low or antibody-binding constants are not high enough. This instant patent aims to identify those antibodies developed by humans that are of a high binding affinity. However, in the case where the most potent natural antibodies against COVID cell surface proteins falls below a specific value that is yet to be determined one must consider engineering Fc fragments to reduce ADE. ADE occurs mostly due to the interactions between the antibody and Fc gamma receptors (FcγRs). In the case of COVID, COVID-19 and mutants ADE may be mediated by the binding of the Fc regions of COVID-bound antibodies to immune cells. ADE may occur in the lumen of the lungs without COVID, COVID-19 or mutants breaching the epithelial lining. Internalization of the antibody bound virus has the potential to promote inflammation and tissue damage through that action or lack thereof of cytokines and chemokines. (See e.g., F. et al., 2008, J. Immunol., 181:6337-6348; Iwasaki, A. & Yang, Y., 2020, Nat. Rev. Immunol. 20:339-341.; Yip, M. S. et al.; 2016, Hong Kong Med. J. 22:25-31; Wang, S. F. et al., 2014, Biochem. Biophys. Res. Commun. 451:208-214).

### Tissue Targeting

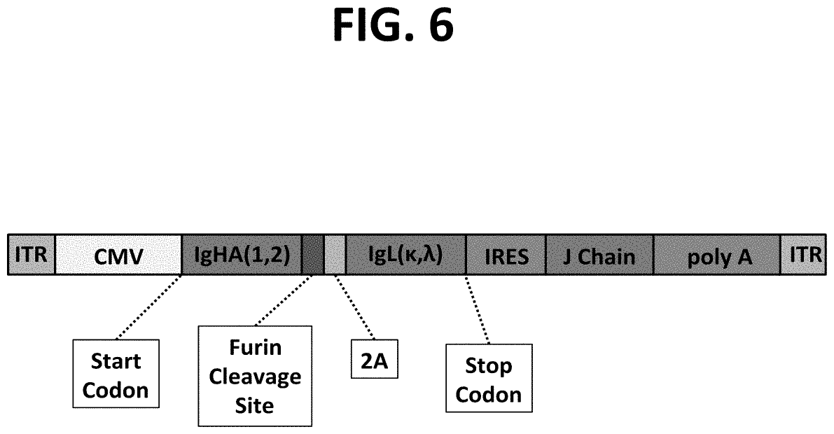

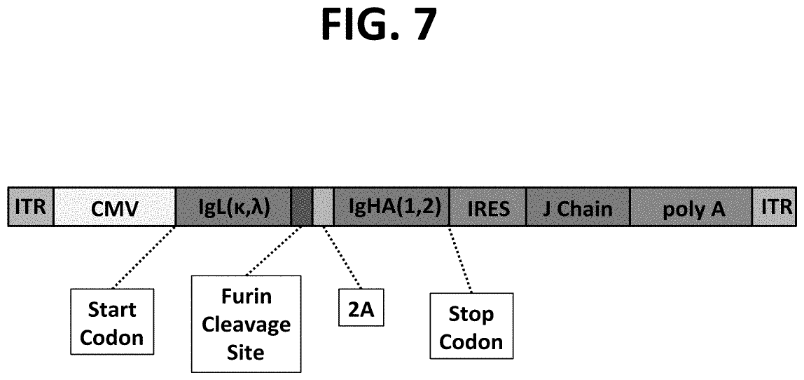

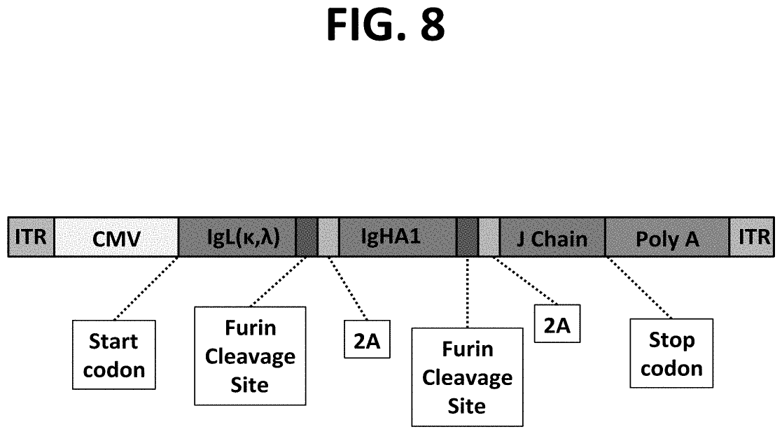

The instant invention contemplates the delivery of the episomally-maintained gene therapy based vaccination/mmunization as an intramuscular injection, intravenous injection or injection proximal to lymph nodes. AAV vectors (See FIGS. 6, 7, 8, 9 and 10) may be delivered intramuscularly where that AAV capsid may transduce the skeletal muscle cell (myocyte) and deliver the vector to muscle nuclei where the vectors may form concatemers or reside as monomeric circular gene elements. Skeletal muscles are particularly beneficial because they are a non-dividing cell population. Thus, episomes not capable of self-replication may persist for years in the skeletal muscle nuclei. It is also possible to deliver naked vector DNA to muscle cells with the use of electroporation. It has been shown that electroporation increase gene transfection efficiency to the muscle nuclei by 100 fold. Electroporation may be defined in this instance as low voltage pulses to the muscle, which allows macromolecules such as DNA vectors to enter the cells more efficiently. (See e.g., Tjelle, T. E., et. al., 2004, Mol. Ther., 9:328-336; Andrews, C. D., et al., 2017, Methods and Clinical Development, 7:74-82)

### Non-Viral Vectors

The vectors of the invention typically include heterologous control sequences, which include, but are not limited to, constitutive promoters, such as the cytomegalovirus (CMV) (SEQ ID NO. 1) immediate early promoter, the RSV LTR, the MOMLV LTR, and the PGK promoter; tissue or cell type specific promoters including mTTR, TK, HBV, haAT, regulatable or inducible promoters, enhancers, 1140 etc. Preferred promoters include the EF1-alpha promoter (Kim et al., Gene 91(2): 217-23 (1990)) and Guo et al., Gene Ther. 3(9):802-10 (1996)). Highly preferred promoters include the elongation factor 1-alpha (EF1a) promoter, a phosphoglycerate kinase-1 (PGK) promoter, a cytomegalovirus immediate early gene (CMV) promoter, chimeric liver-specific promoters (LSPs), a cytomegalovirus enhancer/chicken beta-actin (CAG) promoter, a tetracycline responsive promoter (TRE), a transthyretin promoter (TTR), an simian virus 40 (SV40) promoter and a CK6 promoter. The sequences of these and numerous additional promoters are known in the art. The relevant sequences may be readily obtained from public data-bases and incorporated into vectors for use in practicing the present invention.

In order to express 2 or more proteins in a vector two or more promoters are required. As an example an internal ribosome entry site (IRES) sequence are often used to drive expression of individual genes. Alternatively, an elongation factor 1-alpha promoter (EF1α) may be used. When two genes are linked with an IRES sequence, the expression level of the second gene is often significantly weaker than the first gene (Furler et al., Gene Therapy 8:864-873, 2001). As will be understood by those of skill in the art, expression vectors typically include a promoter operably linked to the coding sequence or sequences to be expressed, as well as ribosome binding sites, RNA splice sites, a polyadenylation site, and transcriptional terminator sequences, as appropriate to the coding sequence(s) being expressed.

### Prolonged Gene Expression

Episomally maintained vectors with self-replicating elements are capable of long-term episomal persistence in human cells and may be referred to as episomally-maintained self-replicating vectors. Typically, these vectors are based on few designs. One common design contains components derived from either EBV or bovine papilloma virus, where there is a greater emphasis for gene therapy related applications with EBV. EBV is a human herpesvirus that can maintain its genome extra-chromosomally as an episome in dividing mammalian cells. Maintenance is also achieved with the EBV viral latent origin of replication oriP and EBV nuclear antigen 1 (EBNA1) that act together to replicate and retain the viral genome in the nucleus. Thus, inclusion of these sequences allows them to be maintained in dividing cells. (See e.g., Conese, M., et. al., 2004, Gene Ther., 11:1735-1741) Although, there are substantial safety and immunotolerance challenges associated with the EBNA1 protein making it unsuitable for present application but future advances may permit their used in clinical trial.

### Vectors for Use in Practicing the Invention

The present invention considers the use of vectors as an efficient and effective means to express immunoglobulins whose polypeptide sequence is derived from memory B-cell with high binding affinity against a viral antigen derived from blood of humans that were infected with a pathogen of interest. In another context only the V-regions (V_{L} and V_{H}) and in some cases portions of the constant regions such as C_{H}1 and C_{L} polypeptide sequence are derived from the immunoglobulin polypeptide sequence of memory B-cells expressing immunoglobulins with high binding affinity against the viral antigen of interest. This strategy provides flexibility to use entire immunoglobulin polypeptide sequences or only V-region sequences in conjunction with other constant region sequences including isotype switched constant region sequences, abbreviated or engineered constant region sequences that cannot be detected by Fc receptors and even a combination of constant region sequences from two sources. E.g. using the C_{H}1 and C_{L} sequences derived from the memory B-cell where the V-region gene elements were identified while using C_{H}2 and C_{H}3 polypeptide sequences from another human source. These strategies collectively allow for both potential improvements in K_{d} values, reduction of the probability that such immunoglobulins will elicit an immune response in a recipient of the episomal vaccine/immunization or the reduced likelihood of antibody dependent enhancement of infection. The pathogenic focuses include COVID, COVID-19 and variants, pandemic pathogens especially respiratory pandemic viruses, and nonpandemic viruses including those that have not been discovered or do not yet exist.

This strategy is undertaken so that at risk individuals and individuals that may not be able to develop adaptive immunity to a virus of interest can be engendered immunity against the pathogen of interest in a relatively short time window in a manner that circumvents their adaptive immune system. Further, because the V-regions of the immunoglobulins are developed by nature and derived from human memory B-cells of human sources that were infected with the pathogen the immunoglobulins coded for by the vectors are more likely to receive FDA approval in more rapid fashion because nature has established safety of the binding regions if not the entire immunoglobulins and effectively serves as the investigation new drug (IND) enabling study.

The vector constructs and viral delivery systems then effectively serve as the mechanism to realize that immunoglobulin based immunity that was discovered in others when considering the expression of IgG1, IgG3, IgA1 and IgA2. However, the present invention further considers the use of vector constructs to express dimeric immunoglobulin A1 that encode for the immunoglobulin heavy chain, the immunoglobulin light chain (kappa or lambda), J-chain and optional use of MZB1 in some embodiments on a single vector construct in order to also engender recipients with a mucosal immunity against the pathogen of interest. In these contexts the present invention considers the use of a variety of vectors to introduce DNA constructs that comprise these coding sequences. The expression of dIgA1 in a single vector construct is important since a mix of different episomes that encode for a polyclonal mix of immunoglobulins potent against the pathogen of interest and likely to prevent mutated form of the pathogen from infecting the recipient of the episomal gene therapy can be rendered less effective if two such gene elements were used to express dIgA1. If dIgA required to vector constructs for encoding its expression such a strategy would require an disproportionately large titer of the vectors containing J-chain and MZB1 to ensure that a sufficient portion of the same cells receive both vectors. However, the intention to express dimeric IgA by distributing the genetic information onto more than one episome is also considered.

Any of a variety of vectors for introduction of constructs (See FIGS. 6, 7, 8, 9 and 10 as possible examples) comprising the coding sequence for immunoglobulins that in some embodiments includes the use of 2A self-cleaving peptide sequences and furin cleavage sites. There are an extensive number of examples of gene expression vectors that are known in the art and such vectors may be viral and non-viral as well as self-replicating or replication deficient. Although, the use of self-replicating vectors to express immunoglobulins while may be employable with lentiviral based gene delivery methods excludes the use of rAAV vectors that have a 4.9 Kb capacity. Non-viral gene delivery methods that may be employed in the practice of the invention include but are not limited to plasmids, liposomes, nucleic acid/liposome complexes and cationic lipids.

Reference to a vector as “recombinant” refers to the linkage of DNA sequences which are not known in nature and which come from two or more organisms in nature. Reference to the “transgene” refers to the gene encoding for a polypeptide in the vector that is intended for some function in the organism that is unrelated to the maintenance or support of the vector function. Expression of the transgene is regulated by sequences that are operatively linked to a polypeptide coding sequence when the expression and/or control sequences regulate the transcription and/or translation of the nucleic acid sequence. Thus expression and/or control sequences can include promoters, enhancers, transcription terminators, polyadenylation sites, replicating elements, a start codon (i.e., ATG) 5′ to the coding sequence, Kozak sequences, splicing signals for introns, a 5′UTR, a 3′UTR and stop codons. (For example, see U.S. Pat. Nos. 7,498,024 B2 and 7,709,224 B2 expressly incorporated by reference herein in their entirety)

Recombinant Adeno Associated Virus (rAAV) vectors are established to exhibit strong transient expression with excellent titer and the ability to enter dividing and non-dividing human cells without eliciting an immune response—upon first time exposure—to the AAV capsid protein structure. However, multiple exposures to an 1265 AAV capsid may elicit an immune response. rAAV vectors delivered via AAV capsids have the ability to enter cells that depends in part on AAV serotype. Further, AAVs are favored for in vivo episomal gene transfer of therapeutic genes because of their low immunogenicity, strong safety record, and high efficiency of transduction of a number of cell types in animal models. (Wu, Z., et. al., 2006, Mol Ther. 14:316-327.) The recombinant vectors of this invention comprise the ability to express dimeric Immunoglobulin A (dIgA), which is a tetramer of heterodimers linked together by disulfide bonds between light and heavy chain immunoglobulins and disulfide bonds between the J-chain protein and the heavy chain immunoglobulin. Further the recombinant vectors of this invention encode for immunoglobulins that are naturally developed in human as part of being naturally exposed to natural forms of a virus. In some instances only a portion—that must include V-region segments of both the light and heavy chain immunoglobulins—of the polypeptide sequence encoding for the immunoglobulins discovered in the blood of individuals infected with the virus of interest will be used in the vector construct. The elements of the recombinant AAV vectors may comprise (A) a packaging site that enables the vector to be placed in replication incompetent AAV virions; (B) the coding sequence for immunoglobulin gene elements e.g. heavy chain (IgH), light chain (IgL), J-chain and associated proteins e.g. MZB1; (C) the optional use of the sequence encoding for a 2A self cleaving peptide site located C-terminal to a furin cleavage site. (D) the optional use of a multi-promoter rAAV vector for immunoglobulin expression. (E) the optional use of both (C) and (D) in a single vector. (See FIGS. 6, 7, 8, 9 and 10 as potential examples) Other elements necessary for or which improve the formation an/or function of the vectors or vector packaging including ITRs. All these methods are well within the practice of those skilled in the art. (For example, see U.S. Pat. Nos. 7,498,024 B2 and 7,709,224 B2)

Adeno-associated virus (AAV) is a helper-dependent human parvovirus, which is able to infect cells latently by chromosomal integration or delivery of an episomally maintain gene element to the nucleus. AAV has significant potential as a human gene therapy vector. For use in practicing the present invention rAAV virions may be produced using standard methodology, known to those of skill in the art and are constructed such that they include, as operatively linked components in the direction of transcription, control sequences including transcriptional initiation and termination sequences, and the coding sequence(s) of interest. More specifically, the recombinant AAV vectors of the instant invention comprise: (1) a packaging site enabling the vector to be incorporated into replication-defective AAV virions; (2) the coding sequence for two or more polypeptides or proteins of interest, e.g., heavy and light chains of an immunoglobulin of interest; (3) the optional use of a sequence encoding a self-processing cleavage site alone or in combination with an additional furin cleavage site. (4) optional use of separate promoters and regulatory elements for each coding sequence (5) Optional use of a combination of (3) and (4). AAV vectors for use in practicing the invention are constructed such that they also include, as operatively linked components in the direction of transcription, control sequences including transcriptional initiation and termination sequences. These components are flanked on the 5′ and 3′ end by functional AAV ITR sequences. By “functional AAV ITR sequences” is meant that the ITR sequences function as intended for the rescue, replication and packaging of the AAV virion. Further, AAV vectors for use in practicing the invention will code for (A) immunoglobulins that are identified from blood, plasma or leukocytes from humans that are or were infected with the pathogen of interest such as COVID (B) immunoglobulins whose V-regions (V_{L} and V_{H}) are derived from immunoglobulins identified from blood, plasma or leukocytes from humans that are or were infected with the pathogen of interest such as COVID (C) immunoglobulins whose antibody binding fragments (Fab) V-regions (V_{L} and V_{H}) and constant heavy region 1 C_{H}1 and constant light constant regions are derived from immunoglobulins identified from the blood, plasma or leukocytes from humans that are or were infected with the pathogen of interest such as COVID. (D) Dimeric Immunoglobulin A (dIgA) that requires DNA coding for the immunoglobulin class A, J chain and optional encoding of MZB1. (For similar examples, see U.S. Pat. Nos. 7,498,024 B2 and 7,709,224 B2; Also see e.g., Fang, J., et al., 2005, Nat. Biotechnol., 23: 584-590.; Krysan, D. J., et. al., 1999, J Biol Chem. 274:23229-23234)

Recombinant AAV (rAAV) vectors are capable of directing the expression and production of selected recombinant polypeptide or protein products in target cells. Thus, the recombinant vectors comprise at least all of the sequences of AAV essential for encapsidation of the vector and the physical structures or inverted terminal repeats

(ITRs) for infection of the recombinant AAV virions. Thus, AAV ITRs for use in the vectors of the invention need not have a wild-type nucleotide sequence (e.g., as described in Kotin, H., 1994, Gene Ther., 5:793-801), and may be altered by the insertion, deletion or substitution of nucleotides or the AAV ITRs may be derived from any of several AAV serotypes. Generally, an AAV vector is a vector derived from an adeno-associated virus serotype, including without limitation, AAV-1, AAV-2, AAV-3, AAV-4, AAV-5, AAV-6, AAV-7, AAV-8, etc. Preferred rAAV vectors have the wild type REP and CAP genes deleted in whole or part, but retain functional flanking ITR sequences that are necessary for the vector to be efficiently encapsulated by the AAV virion. (For example, see U.S. Pat. Nos. 7,498,024 B2 and 7,709,224 B2 expressly incorporated by reference herein in their entirety)

The most widely used platform for producing rAAV involves transfecting Human Embryonic Kidney 293 (HEK293) cells with either two or three plasmids; one encoding the gene of interest, one carrying the AAV rep/cap genes, and another containing helper genes provided by either adeno or herpes viruses which contains the E4, E2a and VA helper genes that mediate AAV replication (See e.g., Janik, J. E., et. al., 1981, “Proc. Natl. Acad. Sci., 78:1925-1929; Buller, R. M. L., et al., 1981, J, Virol. vol. 40:241-24; Matsushita ,T., et. al., 1998, Gene Ther., 5:938-945) The helper construct includes AAV coding regions that are capable of being expressed in the producer cell and which complement the AAV helper function that is absent in the AAV vector. Without such helper genes AAV production is reduced by at least a factor of 10². The helper construct may be designed to down regulate the expression of the large Rep proteins (Rep78 and Rep68), typically by mutating the start codon following p5 from ATG to ACG. (See e.g., as described in U.S. Pat. No. 6,548,286 expressly incorporated by reference herein in their entirety.) This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions encapsulating the recombinant AAV vectors of the instant invention are made by Standard techniques known in the art using AAV packaging cells and packaging technology. Examples of these methods may be found, for example, in U.S. Pat. Nos. 5,436,146, 6,040,183, 6,093,570 and 6,548,286, expressly incorporated by reference herein in their entirety. Further compositions and methods for packaging are described in Wang et al. (US 2002/0168342), expressly incorporated by reference herein in its entirety and include those techniques within the knowledge of those of skill in the art.

In practicing the invention, host cells for producing rAAV virions include mammalian cells, insect cells, microorganisms and yeast. HEK293 is a commonly used host cell that has seen significant success and has become the host cell of choice for AAV production. Host cells can also be packaging cells in which the AAV rep and cap genes are stably maintained in the host cell or producer cells in which the AAV vector genome is stably maintained and packaged. AAV vectors are purified and formulated using techniques known to those familiar in the art.

### Lentiviral Vectors

Lentiviral vectors have been extensively investigated and optimized over the past 20 years (See e.g., Milone, M. C., et al., 2017, Leukemia, 32:1529-1541). Lentiviral vectors may be used in practicing the present invention. Retroviral vectors have been tested and found to be suitable delivery vehicles of genes of interest into the genome of a broad range of target cells. Modified forms of lentiviral vectors may deliver episomes known as integration-deficient lentiviral vectors that are non-viral vectors may be used in the invention (See e.g., Wanisch, K., et al., 2009, The journal of the American Society of Gene Therapy, 17:1316-1332.). One major advantage of lentiviral vectors over AAV vectors is the packaging capacity. The total packaging capacity of an AAV vector is 4.9 kilobases. However, in the instant invention there are vector constructs such as related to the expression of dIgA that involve 3 or 4 transgenes including the immunoglobulin light and heavy chain as well as the J-chain protein and MZB1 that may not be necessary if the non-viral vector is delivered to a B-cell. With 4 transgenes one may not consider the use of a separate promoter for each gene if an AAV capsid is considered as the delivery vehicle. This instant invention considers the expression of dIgA with use of a single open reading frame that can be achieved in the tight space of the AAV non-viral vector. Alternatively, this instant invention considers the use of an intengration-deficient lentiviral vector to deliver the non-viral vector with use of a single open reading frame and up to a separate promoter for each transgene. Lentiviruses have been shown to transduce B-cells of a murine model with persistent expression of the transduced gene. This was accomplished by generating a lentivirus pseudotyped with an anti-CD19 antibody. The lentivirus anti-CD19 antibody targeted the B-cell surface receptor CD19 that mediated endocytosis upon binding the lentivirus to the B-cell, which permitted persistent expression of the transduced gene. (See e.g., Cascalho, M., et al., 2018, Sci Rep, 8:11143) CD19 is present on naïve and memory B-cells. This is relevant in the instant invention that may require B-cells for the expression of dimeric immunoglobulin A (dIgA) as part of engendering mucosal immunity against the virus of interest such as COVID, COVID-19 and its mutants, pandemic and nonpandemic viruses. Lentivirus have seen use in clinical trials and has received FDA approval for immune based gene therapies. (See e.g., Milone, M. C., et al., 2017, Leukemia, 32:1529-1541; Campochiaro, P. A., et al., 2017, Hum Gene Ther. 28: 99-111; Milani, M., et al. 2017, EMBO Mol Med. 9:1558-73). The ability to direct the delivery of lentivirus vectors encoding one or more target protein coding sequences to specific target cells is desirable in practice of the present invention.

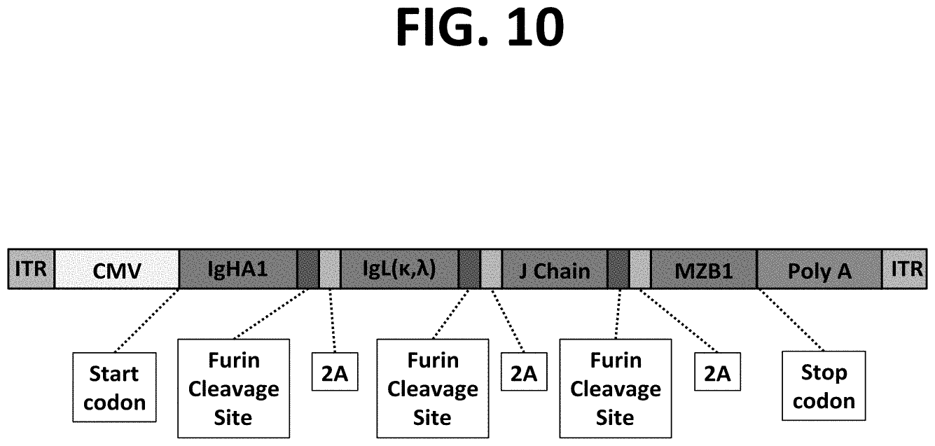

The present invention provides integration-deficient lentiviral vectors comprising one or more transgene sequences and lentiviral packaging vectors comprising one or more packaging elements. (See FIG. 10) Additionally, the present invention provides pseudotyped lentiviral vectors encoding a heterologous or functionally modified envelope protein for producing pseudotyped lentivirus. The lentiviral vectors are intended for episomal expression that may episomally-maintained self-replicating vectors or may lack the gene elements necessary for self-replication.

The term “vector” is used herein to refer to a deoxyribonucleic acid (DNA) molecule capable transferring or transporting another nucleic acid molecule. The transferred nucleic acid is generally linked to, e.g., inserted into, the vector nucleic acid molecule. A vector may include sequences that direct autonomous replication in a cell.

As is evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

Vectors are a common tool for gene delivery (Miller, 2000, Nature. 357: 455-460). Once the virus is integrated into the host genome, it is referred to as a “provirus.”, The provirus serves as a template for RNA polymerase II and directs the expression of RNA molecules, which encode the structural proteins and enzymes needed to produce new viral particles. At each end of the provirus are structures called “long terminal repeats” or “LTRs.”, The term “long terminal repeat (LTR)” refers to domains of base pairs located at the ends of lentiviral DNAs which, in their natural sequence context, are direct repeats and contain U3, R and U5 regions. LTRs generally provide functions fundamental to the expression of lentiviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer-binding site and the R region and contains the polyadenylation sequence. The R (repeat) region is flanked by the U3 and U5 regions. The LTR composed of U3, R and U5 regions and appears at both the 5′ and 3′ ends of the viral genome.

As used herein, the term “packaging signal” or “packaging sequence” refers to sequences located within the lentiviral genome which are required for insertion of the viral RNA into the viral capsid or particle, (See e.g., Clever et al., 1995. J. of Virology, Vol. 69, No. 4; pp. 2101-2109.) Several lentiviral vectors use the minimal packaging signal (also referred to as the psi (Ψ) or (Ψ+) sequence) needed for encapsidation of the viral genome. Thus, as used herein, the terms “psi” and the symbol “Ψ,” are used in reference to the non-coding sequence required for encapsidation of retroviral RNA strands during viral particle formation.

“Self-inactivating” vectors refers to replication-defective vectors, e.g., lentiviral vectors, in which the 3′ LTR enhancer-promoter region, known as the U3 region, has been modified (e.g., by deletion and/or substitution) to prevent viral transcription beyond the first round of viral replication. This is because the 3′ LTR U3 region is used as a template for the left 5′ LTR U3 region during viral replication and, thus, the viral transcript cannot be made without the U3 enhancer-promoter. In a further embodiment of the invention, the 3′ LTR is modified such that the US region is replaced, for example, with a heterologous or synthetic polyA sequence, one or more insulator elements, and/or an inducible promoter. It should be noted that modifications to the LTRs such as modifications to the 3′ LTR, the 5′ LTR, or both 3′ and 5′ LTRs, are also included in the invention.

Integration-deficient lentiviral vectors refers to lentiviral vectors that cannot act as retroviruses. They are nonviral vectors that are episomally-maintained and may or may not be replication competent. To render a lentiviral vector integration deficient the a large part of the U3 region of the 3′LTR and even 5′ LTR are deleted resulting in “dLTR” which eliminates the viral promoter activity and also allowing for the transgene expression to be controlled by the incorporation of an internal promoter additionally the U5 regions may be mutated to eliminated their retroviral function where the LTRs with deleted U3 regions are then referred to as dLTR or 3′-deleted LTRs (dLTR) and 5′-deleted LTRs (dLTR). (See e.g., Apolonia, L., 2009, Ph.D. Thesis, University College London; Yáñez-Muñoz, R. J., et al., 2006, Nat Med. 12:348-353; Conese, M., et. al., 2004, Gene Ther., 11:1735-1741; Karwacz, K., et al., 2009, J. Virol., 83:3094-3103)

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter may be inducible, such that transcription of all or part of the viral genome will occur only when one or more induction factors are present. Induction factors include, but are not limited to, one or more chemical compounds or physiological conditions, e.g., temperature or pH, in which the host cells are cultured.

### U.S. PATENT DOCUMENTS

| | |
|---|---|
| U.S. Pat. No. 5,436,146 A | 7/1995 Shenk et al. |
| U.S. Pat. No. 6,040,183 A | 3/2000 Ferrari et al. |
| U.S. Pat. No. 6,093,570 A | 7/2000 Ferrarietal. |
| U.S. Pat. No. 6,548,286 B1 | 4/2003 Samulski et al. |
| U.S. Pat. No. 7,498,024 B2 | 3/2009 Fang et al. |
| U.S. Pat. No. 7,709,224 B2 | 5/2010 Fang et al. |
| US 2002/0168342 | 11/2002 Wang et al. |

### REFERENCED PUBLICATIONS

Gianchecchi, E., Manenti, A., Kistner, O., Trombetta, C., Manini, I. & Montomoli, E. “Flow to assess the effectiveness of nasal influenza vaccines? Role and measurement of sIgA in mucosal secretions. Influenza Other Respir Viruses.”, vol. 13 No. 5, pp. 429-437, 2019. doi:10.1111/irv.12664
Iwasaki, A. & Yang, Y. “The potential danger of suboptimal antibody responses in COVID-19.”, Nat. Rev. Immunol., vol. 20, pp. 339-341, 2020. https://doi.org/10.1038/s41577-020-0321-6
Pandey, S., & Vyas, G. N. “Adverse effects of plasma transfusion.”, Transfusion, vol. 52, supp. 1 (Suppl 1), pp. 655-795, 2012. https://doi.org/10.1111/j.1537-2995.2012.03663.x
Boe, D. M., Boule, L. A., & Kovacs, E. J. “Innate immune responses in the ageing lung.”, Clinical and experimental immunology, vol. 187, No. 1, pp. 16-25, 2017. https://doi.org./10.1111/cei.12881
Vale, A. M., & Schroeder, H. W., Jr. “Clinical consequences of defects in B-cell development.”, The Journal of allergy and clinical immunology, vol. 125, No. 4, 778-787, 2010. https://doi.org./10.1016/j.jaci.2010.02.018
Pilette, C., Ouadrhiri, Y., Godding, V., Vaerman, J. P., & Sibille, Y. “Lung mucosal immunity: immunoglobulin-A revisited.”, European Respiratory Journal, vol. 18, No. 3, pp. 571-588, 2001. https://erj.ersjournals.com/content/18/3/571
Terauchi, Y., Sano, K., Ainai, A., Saito, S., Taga, Y., Ogawa-Spoto, K., Tamura, S. I., Odagiri, T., Tashiro, M., Fujieda, M., Suzuki, I., & Hasegawa, H. “IgA polymerization contributes to efficient virus neutralization on human upper respiratory mucosa after intranasal inactivated influenza vaccine administration.”, Human vaccines & immunotherapeutics, vol. 14, No. 6, pp. 1351-1361, 2018. https://doi.org/10.1080/21645515.2018.1438791
Melamed, D., Benschop, R. J., Cambier, J. C., & Nemazee, D., “Developmental regulation of B lymphocyte immune tolerance compartmentalizes clonal selection from receptor selection.”, Cell., vol. 92, No. 2, pp.173-182, 1998. https://doi.org/10.1016/s0029-8674(00)80912-5
Wardemann, H., Yurasov, S., Schaefer, A., Young, J. W., Meffre, E., & Nussenzweig, M. C. “Predominant autoantibody production by early human B cell precursors.”, Science, vol. 301, (5638), pp. 1374-1377, 2003. doi:10.1126/science.1086907
Ziegner, M., Steinhauser, G., & Berek, C., “Development of antibody diversity in single germinal centers: selective expansion of high-affinity variants.”, Eur. J. Immunol. Vol. 24, pp. 2393-2400, 1994.
Shlomchik, M. J., Litwin, S., & Weigert, M., “The Influence of Somatic Mutation on Clonal Expansion.”, Prog. in Immunol. Proc. 7^{th} Int. Cong. Immunol. Vol. 7, pp. 415-423, 1990. https://doi.org/10.1007/978-3-642-83755-5_55

Berek, C., & Milstein, C., “Mutation drift and repertoire shift in the maturation of the immune response.”, Immunol Rev. vol. 96, pp. 23-41, 1987. doi:10.1111/j.1600-065x.1987.tb00507.x
Tiller, T., Tsuiji, M., Yurasov, S., Velinzon, K., Nussenzweig, M. C., & Wardemann, H., “Autoreactivity in human IgG+ memory B cells.”, Immunity, vol. 26, No. 2, pp. 205-213, 2007. https://doi.org/10.1016/j.immuni.2007.01.009
Fsuiji, M., Yurasov, S., Velinzon, K., Thomas, S., Nussenzweig, M. C., & Wardemann, H., “A checkpoint for autoreactivity in human IgM+ memory B cell development.”, The Journal of Experimental Medicine vol. 203, No. 2, pp. 393-400, 2006. doi:10.1084/jem.20052033
Wardemann, H., Yurasov, S., Schaefer, A., Young, J. W., Meffre, E., Nussenzweig, M. C. “Predominant autoantibody production by early human B cell precursors.”, Science vol. 30, pp. 1374-1377, 2003. doi:10.1126/science.1086907
Cao, Y., Su, B., Guo, X., Sun, W., Deng, Y., Bao, L., Zhu, Q., Zhang, X., Zheng, Y., & Geng, C., “Potent neutralizing antibodies against SARS-CoV-2 identified by high-throughput single-cell sequencing of convalescent patients'B cells.”, Cell, vol. 182, pp. 73-84, 2020. https://doi.org./10.1016/j.cell.2020.05.025.
Weiss, G. E., Ndungu, F. M., McKittrick, N., Li, S., Kimani, D., Crompton, P. D., Marsh, K., & Pierce, S. K. “High efficiency human memory B cell assay and its application to studying Plasmodium falciparum-specific memory B cells in natural infections.”, Journal of immunological methods, vol. 375, No. 1-2, pp. 68-74. 2012. https://doi.org/10.1016/j.jim.2011.09.006
Hunter, S. A., & Cochran, J. R., “Cell-Binding Assays for Determining the Affinity of Protein-Protein Interactions: Technologies and Considerations.”, Methods Enzymol. vol. 580, pp. 21-44, 2016. doi:10.1016/bs.mie.2016.05.002
Li, P., Selvaraj, P., & Zhu, C. “Analysis of competition binding between soluble and membrane-bound ligands for cell surface receptors.”, Biophysical journal, vol. 77, No. 6, pp. 3394-3406, 1999. https://doi.org/10.1016/50006-3495(99)77171-7
Amanna, I. J., & Slifka, M. K., “Quantitation of rare memory B cell populations by two independent and complementary approaches.”, Journal of immunological methods, vol. 317, No. 1-2, pp. 175-185, 2006. https://doi.org/10.1016/j.jim.2006.09.005
Shen S, et al. (2007) Expression, glycosylation, and modification of the spike (S) glycoprotein of SARS CoV. Methods Mol Biol. 379: 127-35.
Du L, et al. (2009) The spike protein of SARS-CoV—a target for vaccine and therapeutic development. Nat Rev Microbiol. 7 (3): 226-36.
Xiao X, et al. (2004) The SARS-CoV S glycoprotein. Cell Mol Life Sci. 61 (19-20): 2428-3
Setliff, I., Shiakolas, A. R., Pilewski, K. A., Murji, A. A., Mapengo, R. E., Janowska,
K., Richardson, S., Oosthuysen, C., Raju, N., Ronsard, L., et al., “High-Throughput Mapping of B Cell Receptor Sequences to Antigen Specificity.”
Cell, vol. 179, pp.1636-1646, 2019.
Crotty, S., Aubert, R. D., Glidewell, J., & Ahmed, R. “Tracking human antigen-specific memory B cells: a sensitive and generalized ELISPOT system.”, J Immunol Methods., vol. 286, No. 1-2, pp. 111-122, 2004. doi:10.1016/j.jim.2003.12.015
Antonio, E., Muruato, C. R., Fontes-Garfias, P. R., Mariano G.-B., Vineet, D., Menachery, X. X., Pei-Yong, S. “A high-throughput neutralizing antibody assay for COVID-19 diagnosis and vaccine evaluation” bioRxiv 2020.05.21.109546; doi: https://doi.org/10.1101/2020.05.21.109546
Sarzotti-Kelsoe, M., Daniell, X., Todd, C. A., Bilska, M., Martelli, A., LaBranche, C., Perez, L. G., Ochsenbauer, C., Kappes, J. C., Rountree, W., Denny, T. N., & Montefiori, D. C. “Optimization and validation of a neutralizing antibody assay for HIV-1 in A3R5 cells.”, Journal of immunological methods, vol. 409, pp. 147-160, 2014. https://doi.org./10.1016/j.jim.2014.02.013
Klein, U., Rajewsky, K., Kuppers, R., “Human immunoglobulin (Ig)M+IgD+ peripheral blood B cells expressing the CD27 cell surface antigen carry somatically mutated variable region genes: CD27 as a general marker for somatically mutated (memory) B cells.”, The Journal of Experimental Medicine 1905 vol. 188, pp. 1679-1689, 1998.
Vale, A. M., Cavazzoni, C. B., Nobrega, A., & Schroeder, H. W. “The Global Self-Reactivity Profile of the Natural Antibody Repertoire Is Largely Independent of Germline D_{H} Sequence.”, Front. Immunol. vol. 7, No. 296., 2016. doi: 10.3389/fimmu.2016.00296
Nobrega, A., Haury, M., Grandien, A., Malanchere, E., Sundblad, A., & Coutinho, A. “Global analysis of antibody repertoires. II. Evidence for specificity, self-selection and the immunological “homunculus” of antibodies in normal serum.”, Eur J Immunol., vol. 23, No. 11, pp. 2851-2859, 1993. doi:10.1002/eji.1830231119
Haury, M., Grandien, A., Sundblad, A., Coutinho, A., & Nobrega, A. “Global analysis of antibody repertoires. 1. An immunoblot method for the quantitative screening of a large number of reactivities.”, Scand. J. Immunol. vol. 39, No. 1, pp. 79-87, 1994. doi:10.1111/j.1365-3083.1994.tb03343.x
Pritsch, O., Hudry-Clergeon, G., Buckle, M., et al. “Can immunoglobulin C(H)1 constant region domain modulate antigen binding affinity of antibodies?.”, J Clin Invest. Vol. 98. No. 10, pp. 2235-2243, 1996. doi:10.1172/JCI119033
Janda, A., Bowen, A., Greenspan, N. S., Casadevall, A. “Ig Constant Region Effects on Variable Region Structure and Function.”, Front Microbiol. vol. 7 No. 22, 2016. Published 2016 Feb. 4. doi:10.3389/fmicb.2016.00022
Fang, J., Qian, J., Yi, S. et al. “Stable antibody expression at therapeutic levels using the 2A peptide.”, Nat. Biotechnol., vol. 23, pp. 584-590, 2005. https://doi.org./10.1038/nbt1087
Krysan, D. J., Rockwell, N. C., Fuller, R. S. “Quantitative characterization of furin specificity. Energetics of substrate discrimination using an internally consistent set of hexapeptidyl methylcoumarinamides.”, J Biol Chem. vol. 274, No. 33, pp. 23229-23234, 1999. doi:10.1074/jbc.274.33.23229
Lewis, A. D., Chen, R., Montefiori, D. C., Johnson. P. R., Clark, K. R. “Generation of neutralizing activity against human immunodeficiency virus type 1 in serum by antibody gene transfer.”, J Virol. vol. 76, No. 17, pp. 8769-8775, 2002. doi:10.1128/jvi.76.17.8769-8775.2002
Huang, M. T., & Gorman, C. M. “The simian virus 40 small-t intron, present in many common expression vectors, leads to aberrant splicing.”, Molecular and cellular biology, vol. 10, No. 4, pp. 1805-1810, 1990.
Wu, C., & Alwine, J. C. “Secondary structure as a functional feature in the downstream region of mammalian polyadenylation signals.”, Molecular and cellular biology, vol. 24, No. 7, pp. 2789-2796, 2004. https://doi.org./10.1128/mcb.24.7.2789-2796.2004
Lycke, N., Erlandsson, L., Ekman, L., Schön, K., Leanderson, T. “Lack of J chain inhibits the transport of gut IgA and abrogates the development of intestinal antitoxic protection.”, J. Immunol., vol. 163, pp. 913-919, 1999.
Sorensen, V., Sundvold, V., Michaelsen, T. E., Sandlie, I. “Polymerization of IgA and IgM: Roles of Cys309/Cys414 and the secretory tailpiece.”, J. Immunol., vol. 162, pp. 3448-3455, 1999.
Schroeder, H., Wald, D., Greenspan, N., “Chapter 4: Immunoglobulins: Structure and Function”. In Paul, William (ed.). Fundamental Immunology (Book) (6th ed.). Philadelphia, PA: Lippincott Williams & Wilkins. pp. 125-151, 2008. ISBN 978-0-7817-6519-0.
Stadtmueller, B. M., Huey-Tubman, K. E., López, C. J., Yang, Z., Hubbell, W. L., Bjorkman, P. J. “The structure and dynamics of secretory component and its 1970 interactions with polymeric immunoglobulins.”, Elife. vol. 5, e10640, 2016. Published 2016 Mar. 4. doi:10.7554/eLife.10640
Castro, C. D., & Flajnik, M. F., “Putting J chain back on the map: how might its expression define plasma cell development?” Journal of Immunology vol. 193, No. 7, pp. 3248-3255, 2014. https://doi.org/10.4049/jimmunol.1400531
Koshland, M. E., “The coming of age of the immunoglobulin J chain.”, Annu Rev Immunol. vol. 3, pp. 425-453, 1985. doi:10.1146/annurev.iy.03.040185.002233
Bonner, A., Almogren, A., Furtado, P. et al. “Location of secretory component on the Fc edge of dimeric IgA1 reveals insight into the role of secretory IgA1 in mucosal immunity.”, Mucosal Immunol. vol. 2, pp. 74-84, 2009. https://doi.org/10.1038/mi.2008.68
Johansen, F. E., Braathen, R., Brandtzaeg, P. “Role of J Chain in Secretory Immunoglobulin Formation”. Scandinavian Journal of Immunology. vol. 52, No. 3, pp. 240-248, 2000. doi:10.1046/j.1365-3083.2000.00790.x PMID 10972899
Xiong, E., Li, Y., Min, Q., Cui, C., Liu, J., Hong, R., Lai, N., Wang, Y., Sun, J., Matsumoto, R., Takahashi, D., Hase, K., Shinkura, R., Tsubata, T., & Wang, J. Y. “MZB1 promotes the secretion of J-chain-containing dimeric IgA and is critical for the suppression of gut inflammation.”, Proceedings of the National Academy of Sciences of the United States of America, vol. 116, No. 27, pp. 13480-13489, 2019. https://doi.org/10.1073/pnas.1904204116
Yip, M. S. et al. “Antibody-dependent enhancement of SARS coronavirus infection and its role in the pathogenesis of SARS.”, Hong Kong Med. J. vol. 22, pp. 25-31, 2016.
Wang, S. F. et al. “Antibody-dependent SARS coronavirus infection is mediated by antibodies against spike proteins.”, Biochem. Biophys. Res. Commun. vol. 451, pp. 208-214, 2014.
Yasui, F. et al. “Prior immunization with severe acute respiratory syndrome (SARS)-associated coronavirus (SARS-CoV) nucleocapsid protein causes severe pneumonia in mice infected with SARS-CoV.”, J. Immunol., vol. 181, pp. 6337-6348, 2008.
Janeway's Immunobiology, Ninth Edition. 2016. Garland Science: New York, New York. ISBN: (Paperback) 978-0815345053.
Stavnezer, J., & Schrader, C. E. “IgH chain class switch recombination: mechanism and regulation.”, Journal of immunology (Baltimore, Md.: 1950), vol. 193, No. 11, pp. 5370-5378, 2014. https://doi.org/10.4049/jimmunol.1401849
Tjelle, T. E., Corthay, A., Lunde, E., Sandlie, I., Michaelsen, T. E., Mathiesen, I., Bogen, B.,
“Monoclonal antibodies produced by muscle after plasmid injection and electroporation”
Mol. Ther., vol. 9, pp. 328-336, 2004.
Andrews, C. D., Luo, Y., Sun, M., Yu, J., Goff, A. J., Glass, P. J., Padte, N. N., Huang, Y., Ho D. D., “In Vivo Production of Monoclonal Antibodies by Gene Transfer via Electroporation Protects against Lethal Influenza and Ebola Infections Molecular Therapy”—Methods and Clinical Development, vol. 7, No. 15, pp. 74-82, 2017
Kim, et al., “Use of the Human Elongation Factor 1O. Promoter as a Versatile and Efficient Expression System’. Gene, vol. 91, pp. 217-223, 1990.

Guo, et al., “Evaluation of Promoter Strength for Hepatic Gene Expression InVivo Following Adenovirus-Mediated Gene Transfer”, Gene Ther, vol. 3, pp. 802-810, 1996.

Furler, et al., “Recombinant AAV Vectors Containing the Foot-and-Mouth Disease Virus 2A Sequence Confer Efficient Bicistronic Gene Expression in Cultured Cells and Rat Substantia Nigra Neurons’. Gene Ther, vol. 8, pp. 864-873, 2001.
Conese, M., Auriche, C. & Ascenzioni, F. Gene “Therapy Progress and Prospects: Episomally maintained self-replicating systems.”, Gene Ther., vol. 11, pp.1735-1741, 2004. https://doi.org/10.1038/sj.gt.3302362
Kotin, “Prospects for the Use of Adeno-Associated Virus as a Vector for Human Gene Therapy’. Hum. Gene Ther., vol. 5, pp. 793-801, 1994.
Janik, J. E., Huston, M. M., Rose, J. A., “Locations of adenovirus genes required for the replication of adenovirus-associated virus.”, Proc. Natl. Acad. Sci., vol.78 pp. 1925-1929, 1981.
Buller, R. M. L., et al. “Herpes simplex virus types 1 and 2 completely help adenovirus-associated virus replication.”, J Virol vol. 40, pp. 241-24, 1981.
Matsushita ,T., Elliger, S., Elliger, C., et al. “Adeno-associated virus vectors can be efficiently produced without helper virus.”, Gene Ther., vol. 5, No. 7, pp. 938-945, 1998) doi:10.1038/sj.gt.3300680
Milone, M. C., O'Doherty, U. “Clinical use of lentiviral vectors.”, Leukemia. vol. 32, No. 7, pp. 1529-1541, 2017. doi:10.1038/s41375-018-0106-0
Wanisch, K., & Yáñez-Muñoz, R. J., “Integration-deficient lentiviral vectors: a slow coming of age. Molecular therapy.”, The journal of the American Society of Gene Therapy, vol. 17, No. 8, pp. 1316-1332, 2009. https://doi.org./10.1038/mt.2009.122
Campochiaro P. A., Lauer, A. K., Sohn, E. H., Mir, T. A., Naylor S., Anderton, M. C., et al. “Lentiviral vector gene transfer of endostatin/angiostatin for macular degeneration (GEM) study.”, Hum Gene Ther. vol. 28, pp. 99-111, 2017.
Milani M, Annoni A, Bartolaccini S, Biffi M, Russo F, Di Tomaso T, et al. Genome editing for scalable production of alloantigen-free lentiviral vectors for in vivo gene therapy. EMBO Mol Med. 2017;9:1558-73.
Cascalho, M., Huynh, D., Lefferts, A. R. et al. “Durable targeting of B-lymphocytes in living mice.”, Sci Rep, vol. 8, pp. 11143, 2018. https://doi.org/10.1038/s41598-018-29452-0
Wu, Z., Asokan, A., Samulski, R. J., “Adeno-associated virus serotypes: vector toolkit for human gene therapy.”, Mol Ther. vol. 14, pp. 316-327, 2006.
Apolonia, L., “Development and Application of Non-Integrating Lentiviral Vectors for Gene Therapy” Ph.D. Thesis, University College London; London, UK, 2009.

Yáñez-Muñoz, R. J., Balaggan, K. S., MacNeil, A., et al. “Effective gene therapy with nonintegrating lentiviral vectors.”, Nat Med. Vol. 12, No. 3, pp. 348-353, 2006. doi:10.1038/nm1365
Karwacz, K., Mukherjee, S., Apolonia, L., Blundell, M. P., Bouma, G., Escors, D., Collins, M. K.,& Thrasher. A. J., “Nonintegrating lentivector vaccines stimulate prolonged T-cell and antibody responses and are effective in tumor therapy.”, J. Virol. Vol. 83, pp. 3094-3103, 2009.

What is claimed:

1. Episomal expression of monoclonal or polyclonal antibodies (immunoglobulins) of isotypes IgG1, IgG2, IgG3, IgA1 and/or dIgA1 whose polypeptide sequence for light and heavy chains are identified from cells isolated and analyzed according to claim 20 and/or from the blood or plasma of persons that are or were infected with COVID, COVID-19 or COVID mutants and other pandemic viruses.

2. A non-viral vector coding for dimeric immunoglobulin A1 (dIgA1) in claim 1 as IgA1 it was identified from a human cell in claim 1 where the non-viral vector contains the transgenes for (1) immunoglobulin heavy chain of isotype A1 (IgHA1), immunoglobulin light chain that may be kappa or lambda as determined from gene sequencing of the B-cell of interest in claim 1 (IgLκ) or (IgLλ) and J-chain or (2) immunoglobulin heavy chain of isotype A1 (IgHA1), immunoglobulin light chain that may be kappa or lambda as determined from gene sequencing of the B-cell of interest in claim 1 (IgLκ) or (IgLλ), J-chain and Marginal Zone B1 Cell Specific Protein (MZB1). Where (A) The vector comprising in the 5′ to 3′ direction a promoter operably linked to all transgenes expressed as a single open reading frame where each transgene is separated from the subsequent transgene in the 5′ to 3′ direction by (1) a furin cleavage site, (2) a sequence encoding 2A self-processing cleavage site. (B) The vector comprising in the 5′ to 3′ direction the use of separate promoters and regulatory elements for each transgene (C) The vector comprising in the 5′ to 3′ direction any combination of (A) and (B) in a single vector.

3. A non-viral vector coding for any of IgG1, IgG2, IgG3 and IgA1 as it was identified from a human cell in claim 1 where the non-viral vector contains the transgenes for (1) immunoglobulin heavy chain (IgH) and immunoglobulin light chain that may be kappa or lambda as determined from gene sequencing of the B-cell of interest in claim 1 (IgLκ) or (IgLλ). Where (A) The vector comprising in the 5′ to 3′ direction a promoter operably linked to all transgenes expressed as a single open reading frame where each transgene is separated from the subsequent transgene in the 5′ to 3′ direction by (1) a furin cleavage site, (2) a sequence encoding 2A self-processing cleavage site. (B) The vector comprising in the 5′ to 3′ direction the use of separate promoters and polyadenylation elements for each transgene.

4. Construction of non-viral episomal vectors in claims 1, 2 and 3 wherein the vector is selected from the group consisting of an adeno-associated virus (AAV) non-viral vector, an integration-deficient replication-incompetent lentivirus vector, an integration-deficient replication competent adenovirus non-viral vector, a replication deficient adenovirus non-viral vector and a non-viral vector intended to be delivered via a vesicle.

5. Delivery of non-viral vectors in claims 1, 2 and 3 with and AAV capsid, lentivirus viral delivery system or vesicle based delivery system.

6. The vector according to claims 1, 2 and 3 where the sequence encoding the furin cleavage site encodes an oligopeptide with the consensus sequence from a group consisting of RXKRR (SEQ ID NO: 12), RXRYKR (SEQ ID NO: 13), RXRFKR (SEQ ID NO: 14).

7. The vector according to claims 1, 2 and 3 where the 2A self processing cleavage site is from a group consisting of (SEQ ID NO: 15), (SEQ ID NO: 16) and (SEQ ID NO: 17).

8. Administration of gene therapy non-viral vectors in any of claims 1, 2, 3, 9, 10, 11, 13 and/or 14 via intramuscular administration to skeletal muscle, intravenous administration or administration proximal to lymph nodes that may be an intramuscular administration.

9. Episomal expression of monoclonal or polyclonal antibodies (immunoglobulins) whose V-regions (both V_{L} and V_{H}) are identified from cells isolated and analyzed according to claim 20 and/or from the blood or plasma of persons that are or were infected with COVID, COVID-19 or COVID mutants and other pandemic viruses and whose constant regions are of a human source or engineered to improve effector functions. Specifically, an IgG1, IgG2, IgG3, IgA1 or IgA2 immunoglobulin identified from a human blood source to be potent against COVID antigen may be engineered such that the (A) V-regions are conserved but the heavy chain immunoglobulin (IgH) constant regions are replaced with the constant region from a human source that may include a non-divergent well-conserved source such as an IgG1 constant region Cγ₁, IgG2 constant region Cγ₂, IgG3 constant region Cγ₃ or IgA1 constant region Cα₁. (B) Using two human sources coding for mixes of two constant regions from one or two isotypes that may be accomplished with combinations of (1) Fab and natural or engineered Fc domains or (2) F(ab′)2 and natural or engineered pFc′ domains. Where the IgA immunoglobulins are expressed as IgA and/or dIgA through incorporating J-chain into the vector with optional incorporation of MZB1 into the vector.

10. Episomal expression of monoclonal or polyclonal antibodies (immunoglobulins) whose V-regions (both V_{L} and V_{H}) are identified from cells isolated and analyzed according to claim 20 and/or the blood of persons infected with COVID, COVID-19 or mutants or another pandemic pathogen and whose constant regions are engineered to minimize antibody mediated enhancement of infection. An IgG1, IgG3, IgA1 or IgA2 immunoglobulin identified from a human blood source to be potent against COVID antigen may be modified such with the use two human sources coding for the same isotype or mixes of two constant regions from two isotypes that may be 1595 accomplished with combinations of Fab and engineered Fc domains or F(ab′)2 and engineered pFc′ domains of the same or different isotypes. That is either the antibody binding fragment (Fab) or F(ab′)2 regions are conserved. Where the IgA immunoglobulins are both expressed as IgA and dIgA through incorporating J-chain into the vector with optional incorporation of MZB1 into the vector.

11. Episomal expression of monoclonal or polyclonal antibodies (immunoglobulins) whose V-regions (both V_{L} and V_{H}) are identified from cells isolated and analyzed according to claim 20 and/or from the blood of persons infected with COVID, COVID-19 or mutants and whose IgG fragment crystallizable (Fc) or pFc′ regions are of an engineered source that minimizes their binding to Fcγ receptors. An IgG1, IgG2 or IgG3 immunoglobulin identified from a human blood source to be potent against COVID antigen may be modified such that the V-regions are conserved, the C_{H}1, hinge and C_{L} regions are each optionally conserved but the heavy chain fragment crystallizable (Fc) domains or pFc′ domains may be engineered to reduce binding of Fcγ receptors.

12. Construction of non-viral episomal vectors in claims 9, 10, 11, 13 and 14 wherein the vector is selected from the group consisting of an adeno-associated virus (AAV) non-viral vector, an integration-deficient replication-incompetent lentivirus vector, an integration-deficient replication competent adenovirus non-viral vector, a replication deficient adenovirus non-viral vector and a non-viral vector intended to be delivered via a vesicle.

13. A non-viral vector coding for dimeric immunoglobulin A1 (dIgA1) in claims 9 and 10 and also for the expression of any dIgA design unrelated to any other claim in this invention where the non-viral vector contains the transgenes for (1) immunoglobulin heavy chain of isotype A1 (IgHA1), immunoglobulin light chain that may be kappa or lambda as determined from gene sequencing of the B-cell of interest in claim 9 or 10 (IgLκ) or (IgLλ) and J-chain (SEQ ID NO. 11) or (SEQ ID NO. 7) or (2) immunoglobulin heavy chain of isotype A1 (IgHA1), immunoglobulin light chain that may be kappa or lambda as determined from gene sequencing of the B-cell of interest in claim 1 (IgLκ) or (IgLλ), J-chain and Marginal Zone B1 Cell Specific Protein (MZB1) (SEQ ID NO. 8). Where (A) The vector comprising in the 5′ to 3′ direction a promoter operably linked to all transgenes expressed as a single open reading frame where each transgene is separated from the subsequent transgene in the 5′ to 3′ direction by (1) a furin cleavage site, (2) a sequence encoding 2A self-processing cleavage site. (B) The vector comprising in the 5′ to 3′ direction the use of separate promoters and separate polyadenylation elements for each transgene (C) The vector comprising in the 5′ to 3′ direction any combination of (A) and (B) in a single vector.

14. A non-viral vector coding for any of IgG1, IgG2, IgG3 and IgA1 as it is described in claims 9, 10 and 11 where the non-viral vector contains the transgenes for (1) immunoglobulin heavy chain (IgH) and immunoglobulin light chain that may be kappa or lambda as determined from gene sequencing of the B-cell of interest in claim 9, 10 or 11 (IgLκ) or (IgLλ). Where (A) The vector comprising in the 5′ to 3′ direction a promoter operably linked to all transgenes expressed as a single open reading frame where each transgene is separated from the subsequent transgene in the 5′ to 3′ direction by (1) a furin cleavage site, (2) a sequence encoding 2A self-processing cleavage site. (B) The vector comprising in the 5′ to 3′ direction the use of separate promoters and regulatory elements for each transgene.

15. The vector according to claims 1, 2, 3, 9, 10, 11, 13 and 14 wherein the promoter and intermediate promoter is selected from the group consisting of an elongation factor 1-alpha promoter (EF1α) promoter, a phosphoglycerate kinase-1 promoter (PGK) promoter, a cytomegalovirus immediate early gene promoter (CMV), a chimeric liver specific promoter (LSP), a cytomegalovirus enhancer/chicken beta-actin promoter (CAG), a tetracycline responsive promoter (TRE), a transthyretin promoter (TTR), a simian virus 40 promoter (SV40) and a CK6 promoter.

16. The vector according to claims 1, 2, 3, 9, 10, 11, 13 and 14 wherein (A) the intermediate promoter is selected from the group consisting of an elongation factor 1-alpha promoter (EF1α) or internal ribosome entry site (IRES) and (B) the polyandeylation site is selected from a group consisting of simian virus 40 polyadenylation site (SV40 polyA) and Bovine Growth Hormone polyadenylation site (BGH polyA).

17. Any combination of claims 1, 2, 3, 9, 10, 11, 13 and 14. That is polyclonal expression of immunoglobulins may consist of a mix of naturally identified immunoglobulins, artificially modified immunoglobulins and engineered immunoglobulins

18. The vector according to claims 1, 2, 3, 9, 10, 11, 13 and 14 where the sequence encoding the furin cleavage site encodes an oligopeptide with the consensus sequence from a group consisting of RXKRR (SEQ ID NO: 12), RXRYKR (SEQ ID NO: 13), RXRFKR (SEQ ID NO: 14)

19. The vector according to claims 9, 10, 11, 13 and 14 where the 2A self processing cleavage site is from a group consisting of (SEQ ID NO: 15), (SEQ NO: 16) and (SEQ ID NO: 17)

20. Method of processing human blood, plasma or leukocytes to identify and characterizing immunoglobulins expressed by memory B-cells of persons that were infected with a virus of interest that may include COVID, COVID-19 and its mutants, pandemic pathogens and non-pandemic pathogens so DNA coding for them can be incorporated in an episomal expression vector and delivered to recipients as part of a monoclonal of polyclonal immunoglobulin gene therapy based vaccination or immunization. This claim employs the use of assaying memory B-cells from the blood, plasma or leukocytes of person's infected with COVID-19 or mutants by completing a competitive binding coupled with single cell separation assays using a flow cytometry method such as fluorescence activated cell sorting (FACS) with the viral antigen of interest to identify CD27+ IgG and CD27+ IgA memory B-cells with high binding affinities (ideally single digit picomolar to nanomolar dissociation constants (K_{d})) for the antigen of interest. Alternatively, CD27+ IgG and CD27+ IgA memory B-cells may be separated through a competitive binding assay that uses a magnetic pull down methods that utilizes biotinylation between magnetic beeds and an antigen of interest and then subsequently separated by a flow cytometry based method such as FACS as part of a second competitive binding assay between memory B-cells competing for an antigen bound to a flourophore. The result of the two sequential assays will also for the identification of immunoglobulins expressed by high potency memory B-cells for the antigen of interest. Immunoglobulin DNA sequences of the memory B-cells may be obtained from single cell real time polymerase chain reaction (RT-PCR) or nested PCR. Additionally, memory B-cells may be artificially induced to differentiate into plasma secreting cells where somatic hyper mutation will not occur and such secreted antibodies may be assessed by B-cell ELLISPOT and/or ELISA in addition to completing RT-PCR or nested PCR on the antibody-secreting cell. Immunoglobulins will be further assessed using a neutralization assay (NT₅₀) that determines the maximal plasma dilution that allows for a 50% reduction in the relative luminescence (RLU) of a virus reporter vs. background, a panel of self antigen binding assays to ensure the immunoglobulins have no affinity for self further establishing safety and a surface plasmon resonance assays all with the aim of determining their dissociation constant (K_{d}) for the antigen of interest to establish their appropriateness for use in human clinical trials as an antibody gene therapy based vaccine/immunization for the pathogen of interest. This approach allows for more rapid entry of such gene therapies for in human trials and more rapid FDA approvals because the immunoglobulins are established as safe in the human that developed them.
